(19) United States
(12) Patent Application Publication
(10) Pub. No.: US 2025/0325179 A1
(43) Pub. Date: Oct. 23, 2025

(54) HEAD-MOUNTABLE ASSESSMENT DEVICE, AND METHOD OF USING SAME

(71) Applicant: EVOLUTION OPTIKS LIMITED, Christ Church (BB)

(72) Inventors: Raul MIHALI, Westport, CT (US); Khaled EL-MONAJJED, Laval, Quebec (CA)

(21) Appl. No.: 18/705,915

(22) PCT Filed: Nov. 1, 2022
(86) PCT No.: PCT/US2022/048634

§ 371 Date: Apr. 29, 2024

Related U.S. Application Data
(63) Continuation-in-part of application No. PCT/US2022/013564, filed on Jan. 24, 2022.
(60) Provisional application No. 63/200,433, filed on Mar. 5, 2021.
(60) Provisional application No. 63/179,057, filed on Apr. 23, 2021.
(60) Provisional application No. 63/274,873, filed on Nov. 2, 2021.
(60) Provisional application No. 63/274,873, filed on Nov. 2, 2021.
(60) Provisional application No. 63/363,016, filed on Apr. 14, 2022.

(51) Int. Cl. A61B 3/113; A61B 3/00; A61B 5/00

(52) U.S. Cl. CPC A61B 3/113; A61B 3/0041; A61B 3/0091; A61B 5/4064; A61B 5/4088; A61B 5/4863; A61B 5/6803

(57) ABSTRACT

A head-mountable device for performing an oculomotor assessment of a user. the head-mountable device comprising a mechanically displaceable oculomotor stimulus. an actuation mechanism operable to displace the mechanically displaceable oculo-motor stimulus relative to the user so to present the mechanically displaceable oculomotor stimulus to the user in accordance with the oculomotor assessment, and an eye tracking system configured to monitor an oculomotor response of the user to the mechanically displaceable oculomotor stimulus. The device further comprises a digital data processor in communication with the eye tracking system and operable to execute digital instructions for recording the oculomotor response to the mechanically displaceable stimulus presented in accordance with the oculomotor assessment.

### CROSS REFERENCE TO RELATED APPLICATIONS

This application is a continuation-in-part of PCT/US2022/013564 filed Jan. 24, 2022, which claims priority to U.S. Provisional Application No. 63,200,433 filed Mar. 5, 2021, U.S. Provisional Application No. 63/179,057 filed Apr. 23, 2021, and U.S. Provisional Application No. 63/274,873 filed Nov. 2, 2021, the entire content of each of which is incorporated herein by reference.

This application also claims priority to U.S. Provisional Application No. 63/363,016 filed Apr. 14, 2022, and U.S. Provisional Application No. 63/274,873 filed Nov. 2, 2021, the entire content of each of which is incorporated herein by reference.

### FIELD OF THE DISCLOSURE

The present disclosure relates to vision-based assessments, and, in particular, to a head-mountable assessment device, and method of using same.

### BACKGROUND

The oculomotor system is a relatively accessible portion of the nervous system, wherein abnormalities in its behaviour may serve as biomarkers for a range of conditions. For example, a traumatic brain injury such as a concussion may result in visual disorders related to convergence insufficiency (CI), accommodative insufficiency (AI), and mild saccadic dysfunction (SD). It may also be associated with abnormalities of saccades, pursuit eye movements, convergence, accommodation, and the vestibular-ocular reflex. Accordingly, evaluation of one or more of these aspects may be useful in the assessment of cognitive function of an individual.

Further, while it was once assumed that the hallmark of a concussion was a loss of consciousness, recent evidence suggests that a majority of people diagnosed with a concussion did not actually experience this symptom. Rather, diagnosis of a traumatic brain injury (TBI) is notoriously challenging, in part due to variability of associated symptoms and the severity thereof. For instance, measurements of TBI severity are typically assessed using a CT structural imaging scan, and/or assessment of the level of consciousness of a patient and the duration of post-traumatic amnesia. Evaluation of severity may then be assessed on a number of scales, such as the Glasgow coma score (CGS). Further, a concussion is a form of TBI that may be considered a functional, rather than a structural injury. In some cases, tissue damage resulting from a jolt to the head may bruise blood vessels, resulting in tissue damage and chemical variations that may degrade information processing throughout the brain, which ultimately may affect sensorimotor functions.

Recent evidence further suggests that oculomotor behaviour may serve as a biomarker in the assessment of a potential TBI. For instance, up to 80% of concussed athletes show some eye dysfunction. The oculomotor system being a relatively accessible portion of the nervous system, assessment of eye function may thus provide valuable information in the evaluation of the presence or degree of cognitive impairment. For example, after a concussion, common ensuing visual disorders may include convergence insufficiency (CI), accommodative insufficiency (AI), and mild saccadic dysfunction (SD). Since a mild concussion is frequently associated with abnormalities of saccades, pursuit eye movements, convergence, accommodation, and the vestibular-ocular reflex, evaluating the vision system of an individual suspected of being cognitively impaired with respect to one or more of these aspects may prove useful in the early diagnosis and/or categorisation of a TBI. Further, such assessment may not only relate to the assessment of a concussion or post-concussion syndrome (PCS), but also to a host of other cognitive impairments, such as autism, PTSD, or schizophrenia, to name a few.

Oculomotor behaviour is typically categorised based in part on the relative amounts of activity observed in respective portions of the brain. For instance, fixations typically involve maintaining eye position in a relatively still state in order to hold the image of a stationary target on the fovea, giving rise to a high degree of visual acuity. Smooth pursuits, on the other hand, relate to the tracking of a moving stimulus to stabilise an image on the fovea. These may be considered a two-phase process, wherein initiation relates to the movement of the eye while no information is recorded, and maintenance relates to the recording of an internal representation of the target in motion as the brain updates and enhances performance of the pursuit of the moving target. Of particular interest due to their relationship with cognitive and motor processes, another category of oculomotor behaviour comprises saccades, which relate to the rapid movement of the fovea between two fixation points, and are often characterised by a velocity, duration, accuracy, and initiation time.

Accordingly, self-paced saccadic eye movements, for instance, have been designated as a potential biomarker for some brain-related injuries, such as post-concussion syndrome, and may be monitored to assess a recovery status associated therewith. Further, various multidimensional methods have been proposed to detect and characterise sensorimotor deficits associated with TBI. For instance, there has been demonstrated a link between higher order visual perception/cognition and eye movements, which may be related to impairment and/or reduction in accuracy, precision, and speed of information processing within cortical circuits. To name one example, Liston et al. (Liston D B, Wong L R. Stone L S., ‘Oculometric Assessment of Sensorimotor Impairment Associated with TBI’, Optom Vis Sci. 2017; 94 (1): 51-59) found that some individuals having experienced a TBI reported a degradation of oculometrics, such as pursuit latency, initial pursuit acceleration, pursuit gain, catch-up saccade amplitude, proportion smooth tracking, or speed responsiveness. In another example, Hunfalvay et al. (Hunfalvay, M, et al., ‘Horizontal and Vertical Self-Paced Saccades as a Diagnostic Marker of Traumatic Brain Injury’, Concussion 2019; 4 (2): CNC60) established eye tracking tests to measure horizontal and vertical saccades as a proxy for neural deficits, finding that individuals with a concussion were correctly identified 77% and 64% of the time, respectively, while similar results were achieved for the identification of individuals without a concussion.

Various eye tracking assessment algorithms have been proposed to monitor or diagnose brain injuries. For instance, U.S. patent application Ser. No. 18/0,235,532 entitled ‘Method and System for Detection Concussion’ and published to Samandani, et al. on Aug. 23, 2018 discloses a method for identifying a concussion through the analysis of a subject's blinking as compared to a baseline. Similarly, Oculogica's EyeBOX®, a device marketed for concussion assessment, provides a BOX ScoreSM based on similar eye tracking analysis.

U.S. patent application Ser. No. 19/0,239,790 entitled ‘Systems and Methods for Assessing User Physiology Based on Eye Tracking Data’ and published to Gross and Hunfalvay on Aug. 8, 2019 discloses another example of a method of assessing user physiology based on eye tracking. Such processes may be used to provide reports reflective of potential neurological problems, such as those generated by the RightEye EyeQ™ technology.

Digital eye tracking tests such as Hunfalway, et al., as well as other similar approaches, may offer a degree of precision and analysis that comprise an improvement over conventional ‘follow my finger’ tests performed by a neurologist or neuro-optometrist. For instance, the FDA has approved the RightEye™ eye tracking system as a means of recording and analysing eye movements as a patient tracks stimuli displayed on a 2D screen to support identification of visual tracking impairments. Similarly, the FDA-approved EyeBOX® by Oculogica®, and EYE-SYNC® by SyncThink™, track eye movements as a patient follows objects on a display screen to assist in the assessment of

TBI, the latter providing a head-mounted display connected to a computer tablet. While such systems may provide the ability to perform some oculomotor tests related to the assessment of potential cognitive impairments, such as concussions, with a relatively high degree of accuracy, patients are typically restricted to tracking objects at a fixed distance in two dimensions. Various other important oculomotor assessments, however, such as those related to convergence, may require a depth component, wherein the object to be tracked or focused on changes depth planes, or moves towards or away from the eyes of the subject. While such assessments have been contemplated using a head-mounted display, for instance in U.S. patent application Ser. No. 19/0,082,954 entitled ‘Objective Testing of Vergence Dysfunction for Diagnosis and Vergence Recovery Convalescence Using Dynamic Vergence Testing Platform Including 3D Head Mounted Display System with Integrated Eye Tracking Technology’ published Mar. 21, 2019 to Kiderman and Ashmore, such systems continue to rely on 2D displays that attempt to trick the visual system of the user into perceiving a change in object depth by presenting stimuli to be tracked in the context of background stimuli.

Similarly, U.S. Pat. No. 9,004,687 entitled ‘Eye Tracking Headset and System for Neuropsychological Testing Including the Detection of Brain Damage’ and issued to Stack on Apr. 14, 2015 discloses a headset operable to display 2D images for performing smooth pursuit eye tracking exams to indicate potential cognitive impairment. Conversely, U.S. patent application Ser. No. 19/0,082,954 entitled ‘Objective Testing of Vergence Dysfunction for Diagnosis and Vergence Recovery Convalescence Using Dynamic Vergence Testing Platform Including 3D Head Mounted Display System with Integrated Eye Tracking Technology’ published Mar. 21, 2019 to Kiderman and Ashmore discloses a wired head-mounted display to perform vergence dysfunction tests that attempts to simulate a change in perceived object depth using on-screen depth cues.

Taking this notion one step further, U.S. Pat. No. 10,719,992 entitled ‘Augmented Reality Display System for Evaluation and Modification of Neurological Conditions, Including Visual Processing and Perception Conditions’ and issued to Samec, et al. on Jul. 21, 2020 further attempts to mimic the effects of light originating from an object at a given depth, while attempting to improve the accommodation-vergence reflex, by manipulating the divergence properties of light emanating from waveguides in augmented reality display goggles that transmit light from an external environment. Such AR systems, however, are not ideal for cognitive impairment testing. For instance, various tests benefit from an isolated viewing environment that is, for instance, controlled, and/or free of distraction, which otherwise may influence eye movement, or assessment thereof. Further, the manipulation of the divergence properties of light from waveguides is not particularly well suited to providing a range of perceived depth planes, or a sufficient quality of displayed content (e.g. resolution), to adequately perform a cognitive assessment.

Indeed, various challenges are known to exist with respect to the provision of visual content using augmented reality (AR) and virtual reality (VR) systems. For example, conflicting sensory stimuli experienced by a user of an AR or VR system may lead to user fatigue or nausea. U.S. Pat. No. 10,871,627 entitled ‘Head-Mounted Display Device with Direct-Current (DC) Motors for Moving Displays’ and issued to Fang, et al. on Dec. 22, 2020 attempts to address this issue by coupling a DC motor to a display of an AR or VR system, thereby allowing the display to move during use and mitigate vergence-accommodation conflicts.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a head-mountable assessment device, and method using same, that overcome some of the drawbacks of known techniques, or at least, provides a useful alternative thereto. Some aspects of this disclosure provide examples of such systems and methods.

In accordance with one aspect, there is provided a head-mountable device for performing an oculomotor assessment of a user, the head-mountable device comprising a mechanically displaceable oculomotor stimulus, an actuation mechanism operable to displace the mechanically displaceable oculomotor stimulus relative to the user so to present the mechanically displaceable oculomotor stimulus to the user in accordance with the oculomotor assessment, and an eye tracking system configured to monitor an oculomotor response of the user to the mechanically displaceable oculomotor stimulus. The device further comprises a digital data processor in communication with the eye tracking system and operable to execute digital instructions for recording the oculomotor response to the mechanically displaceable stimulus presented in accordance with the oculomotor assessment.

In one embodiment, the digital data processor is further in communication with the actuation mechanism and is operable to execute digital instructions for performing the oculomotor assessment by digitally activating the actuation mechanism to present the mechanically displaceable oculomotor stimulus to the user in accordance with the oculomotor assessment.

In one embodiment, the actuation mechanism comprises a manually manipulatable actuation mechanism comprising a user-manipulatable component which, upon manual displacement by the user, elicits a corresponding displacement of the mechanically displaceable oculomotor stimulus.

In one embodiment, the actuation mechanism is operable to translate the mechanically displaceable oculomotor stimulus in a direction towards or away from the user's eyes.

In one embodiment, the oculomotor assessment comprises one or more of a vergence response assessment, a convergence insufficiency assessment, or an accommodation assessment.

In one embodiment, the mechanically displaceable oculomotor stimulus comprises a fixation stick mechanically coupled with the actuation mechanism.

In one embodiment, the actuation mechanism comprises one or more of an electric motor, a pulley, a belt, a cable, a threaded rod, a hydraulic or pneumatic actuator, or a cog.

In one embodiment, the mechanically displaceable oculomotor stimulus is pivotably coupled with the actuation mechanism to provide the mechanically displaceable oculomotor stimulus in a retracted configuration or an extended configuration.

In one embodiment, the actuation mechanism is operable to mechanically configure the mechanically displaceable oculomotor stimulus in one or more of the retracted configuration or the extended configuration.

In one embodiment, the device further comprises a retaining structure configured to maintain the mechanically displaceable oculomotor stimulus in the retracted configuration upon the actuation mechanism displacing the mechanically displaceable oculomotor stimulus to a stimulus retaining position.

In one embodiment, the extended configuration is provided by a force upon the actuation mechanism displacing the mechanically displaceable oculomotor stimulus to a stimulus presentation position.

In one embodiment, the device further comprises a digital display screen viewable by the user at a designated distance from the user's eyes, wherein the digital display screen is operable to render a complementary two-dimensional oculomotor stimulus at the designated distance in accordance with a complementary oculomotor assessment.

In one embodiment, the mechanically displaceable oculomotor stimulus is retractable to provide the user an unimpeded line of sight to the complementary two-dimensional oculomotor stimulus.

In one embodiment, the digital display screen comprises a widescreen display to render the complementary two-dimensional oculomotor stimulus horizontally displaceable in a wide binocular field of view to stimulate a complementary wide field of view oculomotor response thereto in accordance with the complementary assessment.

In one embodiment, the device further comprises a head-mountable housing that, when mounted against the user's face, defines an internal visual stimulation chambre therein for presenting an oculomotor stimulus.

In one embodiment, the device further comprises an illumination source to illuminate the mechanically displaceable oculomotor stimulus so to be perceivable by the user.

In one embodiment, the device further comprises an external assessment indicator externally disposed on the head-mountable housing and configured to output an indicator signal representative of one or more of an assessment status or a screening indicator corresponding with a health risk associated with the oculomotor assessment.

In one embodiment, the device further comprises an operator application digitally executable on a distinct operator device having an operator display screen and a communication interface to the head-mountable device, wherein the operator application comprises digitally executable instructions to render a graphical user interface (GIU) on the digital display screen and receive as input therefrom manual digital control of the mechanically displaceable oculomotor stimulus such that a stimulus displacement of the mechanically displaceable oculomotor stimulus corresponds with a manual displacement entered via the GUI.

In one embodiment, the mechanically displaceable oculomotor stimulus comprises an indicium perceptible to the user and associated with the oculomotor assessment.

In one embodiment, the device further comprises a displacement sensor configured to monitor a stimulus displacement during the oculomotor assessment.

In one embodiment, the displacement sensor comprises a potentiometer.

In one embodiment, the digital data processor is further operable to execute digital instructions for outputting an assessment response signal representative of the oculomotor response.

In accordance with another aspect, there is provided a head-mountable device for performing a vision-based assessment of a user, the head-mountable device comprising a head-mountable housing that, when mounted against the user's face, defines an internal visual stimulation chambre therein, a mechanically displaceable visual stimulus disposed within the internal visual stimulation chambre and configured to be translated relative to the head-mountable housing in accordance with the vision-based assessment, and an actuation mechanism operable to displace the mechanically displaceable visual stimulus relative to the user so to present the mechanically displaceable visual stimulus to the user in accordance with the vision-based assessment.

In one embodiment, the device further comprises an internal eye tracker to monitor a user eye response to said mechanically displaceable visual stimulus.

In one embodiment, the visual stimulus is at least one of manually or electronically actuated.

In one embodiment, the actuation mechanism comprises a manually manipulatable actuation mechanism which, upon manual displacement, elicits a corresponding displacement of said mechanically displaceable visual stimulus.

In one embodiment, the actuation mechanism is operable to translate said mechanically displaceable visual stimulus in a direction towards or away from the user's eyes.

In one embodiment, the vision-based assessment comprises one or more of a vergence response assessment, a convergence insufficiency assessment, or an accommodation assessment.

In one embodiment, the mechanically displaceable visual stimulus comprises a fixation member mechanically coupled with said actuation mechanism.

In one embodiment, the fixation member is retractably coupled to said actuation mechanism to provide said mechanically displaceable visual stimulus in a retracted configuration or a deployed configuration as the actuation mechanism is actuated.

In one embodiment, the device further comprises a retaining structure configured to maintain said mechanically displaceable visual stimulus in said retracted configuration upon said actuation mechanism displacing said mechanically displaceable visual stimulus to a stimulus retaining position.

In one embodiment, the deployed configuration is provided by a force upon said actuation mechanism displacing said mechanically displaceable visual stimulus to a deployed stimulus presentation position.

In one embodiment, the device further comprises a digital display screen mounted within said housing for viewing by the user, wherein said digital display screen is operable to render a complementary two-dimensional vision-based stimulus.

In one embodiment, the mechanically displaceable visual stimulus is retractable to provide the user an unimpeded line of sight to said complementary two-dimensional vision-based stimulus.

In one embodiment, the digital display screen comprises a widescreen display to render said complementary two-dimensional vision-based stimulus horizontally displaceable in a wide binocular field of view to stimulate a complementary wide field of view response thereto in accordance with said complementary assessment.

In one embodiment, the device further comprises a displacement sensor configured to monitor a stimulus displacement during the vision-based assessment.

In one embodiment, the displacement sensor comprises a potentiometer.

In accordance with another aspect, there is provided a video-nystagmograph (VNG) wearable by a user, comprising: a head-mountable digital display for selectively rendering visual content to the user in accordance with a designated ocular test; a displaceable visual stimulus selectively displaceable toward and away from said digital display in providing a variable depth visual stimulus to the user in accordance with a complementary ocular test; and an eye tracker.

In one embodiment, the VNG further comprises a head-mountable housing that, when mounted against the user's face, defines an internal visual stimulation chambre therein, at the end of which is disposed said digital display and within which said displaceable visual stimulus is displaceable.

In one embodiment, the VNG further comprises an actuation mechanism operable to mechanically displace said displaceable visual stimulus relative to the user.

In one embodiment, the actuation mechanism comprises at least one of a manually or an electronically actuated mechanism.

In one embodiment, the complementary ocular test comprises one or more of a vergence response assessment, a convergence insufficiency assessment, or an accommodation assessment.

In one embodiment, the displaceable visual stimulus comprises a fixation member mechanically displaceable relative to the user.

In one embodiment, the fixation member is selectively retractable and deployable.

In one embodiment, the VNG further comprises a displacement sensor configured to monitor a stimulus displacement during said complementary ocular test.

In accordance with another aspect, there is provided a method of performing an oculomotor assessment of a user, the method comprising providing the user with a head-mountable device substantially as herein described, and, via the actuation mechanism thereof, displacing the mechanically displaceable oculomotor stimulus in accordance with the oculomotor assessment.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIGS. 1A to 1C are s cross-sectional views of an exemplary head-mountable mechanism comprising a mechanically displaceable oculomotor stimulus in various configurations, in accordance with one embodiment;

FIG. 2A is a side view of an exemplary head-mountable device comprising a mechanically displaceable oculomotor stimulus;

FIG. 2B is a rear perspective view of various components of an exemplary actuation mechanism for the mechanically displaceable oculomotor stimulus of the device of FIG. 2A;

FIG. 2C is a top perspective view of various components of the exemplary manual actuation mechanism for the mechanically displaceable oculomotor stimulus of the device of FIG. 2A;

FIG. 2D is a side view of various components of the exemplary manual actuation mechanism for the mechanically displaceable oculomotor stimulus of the device of FIG. 2A;

FIG. 2E is a top perspective view of an alternate arrangement of components to that illustrated in FIG. 2C;

FIGS. 3A and 3B are different perspective views of an exemplary electronic actuation mechanism for displacing a mechanically displaceable oculomotor stimulus in a head-mountable device, in accordance with one embodiment;

FIG. 4 is a perspective view of an alternative actuation mechanism for displacing a mechanically displaceable oculomotor stimulus in a head-mountable device, in accordance with one embodiment;

FIG. 5A is a rear-side perspective view another exemplary head-mountable device comprising a mechanically displaceable oculomotor stimulus, when mounted to a user's head;

FIG. 5B is a rear view of internal components of the head-mountable device of FIG. 5A, showing the mechanically displaceable oculomotor stimulus in a fully deployed forward configuration;

FIG. 6A is a perspective view the internal components as shown in FIG. 5B, showing the mechanically displaceable oculomotor stimulus thereof in a retracted configuration;

FIG. 6B is a cross-sectional perspective view of the internal components of FIG. 6A taken along line B-B thereof;

FIG. 7A is a perspective view the internal components as shown in FIG. 5B, showing the mechanically displaceable oculomotor stimulus thereof in a partially deployed configuration;

FIG. 7B is a cross-sectional perspective view of the internal components of FIG. 7A taken along line B-B thereof;

FIG. 8A is a perspective view the internal components as shown in FIG. 5B, showing the mechanically displaceable oculomotor stimulus thereof in a fully deployed rearward configuration;

FIG. 8B is a cross-sectional perspective view of the internal components of FIG. 8A taken along line B-B thereof;

FIG. 9A is a perspective view the internal components as shown in FIG. 5B, showing the mechanically displaceable oculomotor stimulus thereof in a fully deployed forward configuration;

FIG. 9B is a cross-sectional perspective view of the internal components of FIG. 9A taken along line B-B thereof; and

FIGS. 10A to 12B are schematics illustrating various exemplary configurations of mechanically displaceable visual stimuli to perform various exemplary oculomotor assessments using various exemplary HMD configurations, in accordance with various embodiments.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as ‘configured to’ perform one or more functions or ‘configured for’ such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of ‘at least one of X, Y, and Z’ and ‘one or more of X, Y and Z’ may be construed as X only, Y only, Z only, or any combination of two or more items X, Y, and Z (e.g., XYZ, XY, YZ, ZZ, and the like). Similar logic may be applied for two or more items in any occurrence of ‘at least one . . . ’ and ‘one or more . . . ’ language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase ‘in one of the embodiments’ or ‘in at least one of the various embodiments’ as used herein does not necessarily refer to the same embodiment, though it may. Furthermore, the phrase ‘in another embodiment’ or ‘in some embodiments’ as used herein does not necessarily refer to a different embodiment, although it may. Thus, as described below, various embodiments may be readily combined, without departing from the scope or spirit of the innovations disclosed herein.

In addition, as used herein, the term ‘or’ is an inclusive ‘or’ operator, and is equivalent to the term ‘and/or,’ unless the context clearly dictates otherwise. The term ‘based on’ is not exclusive and allows for being based on additional factors not described, unless the context clearly dictates otherwise. In addition, throughout the specification, the meaning of ‘a,’ ‘an,’ and ‘the’ include plural references. The meaning of ‘in’ includes ‘in’ and ‘on.’

The term ‘comprising’ as used herein will be understood to mean that the list following is non-exhaustive and may or may not include any other additional suitable items, for example one or more further feature(s), component(s) and/or element(s) as appropriate.

As noted above, while various advances have been made in the development of tools and techniques for assessing or screening for concussions, or similar brain injuries, the availability of reliable, comfortable and accessible solutions remains limited. Indeed, while some head-mountable solutions have been proposed using 2D displays or A/R/V/R solutions, their practicality and utility remain limited.

In accordance with some embodiments, a head-mountable assessment device is described herein as comprising a mechanically displaceable visual (e.g. oculomotor) stimulus that can be actuated so to trigger a visual (e.g. oculomotor) response in the wearer of the device, such that such response can be observed and evaluated for cognitive impairment assessment and/or screening. Indeed, in some such embodiments, a visual stimulus can be mechanically displaced toward and away from the user's eyes to trigger a corresponding eye response similar to that triggered by a medical practitioner in the field requesting that a patient visually follow the fore and aft movement of a physical object like a finger or a pen, for example.

As will be detailed further below, the mechanically displaceable stimulus can be deployed within a head-mountable housing that, when mounted against a user's face, defines an internal (visually isolating) visual stimulation chamber. An eye tracking system, for example, may be incorporated within the head-mountable housing to track an oculomotor response, or other eye or vision-based responses, using one or more cameras, optical probes, gaze trackers, or like eye-tracking systems known in the art.

In yet further embodiments, the mechanically actuated visual stimulus may be combined with a 2D display, or again a 3D light field display, to provide complementary testing, screening and/or assessments, again optionally tracked using onboard eye-tracking systems. For example, the mechanically actuated stimulus may be selectively deployed within a combined head-mountable device and retracted so to selectively permit unobstructed viewing of a combined 2D or 3D display, thereby allowing for an increasing range of testing and/or assessment capabilities.

For example, in some embodiments, the mechanically actuated visual stimulus can be selectively deployed and used to track an oculomotor response to a mechanical stimulus (e.g. a physical object such as a fixation stick) being mechanically actuated to move fore and aft within a head-mountable visualization chamber, and retracted so to permit complementary assessments via an on-board widescreen display disposed at the back of the head-mountable housing and chamber, thus in concert providing a range of 2D and 3D assessment options.

In yet other examples, a light field display can offer the ability to directly control the image plane or depth at which content is virtually located and/or perceived. Accordingly, a light field display system equipped with, for instance, eye tracking functionality, may enable assessment of oculomotor behaviour, not only as it pertains to the performance of two-dimensional tests, as described above, but also to three-dimensional assessments that may be more ‘true-to-life’ than those provided from static 2D or augmented reality display systems that rely on tricking the visual system with depth cues. For example, Applicant's co-pending U.S. Patent Application No. 63/179,057 entitled ‘Cognitive Impairment Testing System, and Method Using Same’, the entire contents of which are hereby incorporated by reference, discloses light field-based systems and methods operable to perform a wide range of cognitive impairment assessments, including both 2D and 3D tests for evaluating the oculomotor system of a user. While such light field-based systems and methods offer a means for performing various ocular assessments, such as vergence, saccadic, and pursuit tests, the ability to create a light field requires, in addition to a 2D display screen, various optical components, such as lenses, microlens arrays or like light field shaping elements, and significant processing resources to render ray-traced content. Such components generally add weight and complexity, increase costs, and reduce transportability of head-mounted devices. Such considerations thus come into play when opting for one or another of the embodiments described herein.

Accordingly, as noted above, some of the systems and methods described herein provide, in accordance with different embodiments, different examples for presenting, while user gaze is monitored and within a single head-mounted device, various stimuli disposed or moving in up to three dimensions, without requiring the generation of a light field (e.g. using unrefracted light). Accordingly, various systems or devices as herein described may provide stimuli for various 1D, 2D, or 3D assessments and/or exercises with fewer optical components and processing resources, and less circuitry than is required for conventional light field display systems. However, it will be appreciated that various embodiments herein described may additionally or alternatively provide for a system or method employing a head-mounted display for performing light field-based assessments while providing improvements over conventional light field systems. For instance, various embodiments provide solutions to challenges associated the inherent complexity, bulk, weight, and lack of transportability of light field systems, and provide improved dynamic range as compared to traditional light field systems with respect to the presentation of visual content, and the range and ultimate performance of assessments.

In accordance with various embodiments, an assessment system or device, or a method using same, as herein described, may comprise a head-mounted device or head-mountable device (HMD) that may provide a medical practitioner with quantitative metrics pertaining to eye and head dynamics as the wearer of the HMD (the ‘user’) is directed to follow with their eye(s) a stimulus or stimuli presented in accordance with a designated pattern or sequence. In accordance with some embodiments, such a pattern or sequence may correspond with a 1D, 2D, or 3D oculomotor assessment or exercise. Such data may then be used by the practitioner for subsequent analysis to, for instance, inform decisions or practices with respect to the user.

For example, and without limitation, an HMD as herein described may be employed to screen for or assess a variety of cognitive functions or conditions, non-limiting examples of which may include TBI, attention deficit hyperactivity disease, Alzheimer's disease, Parkinson's disease, Tourette's syndrome, progressive supranuclear palsy, or motor neuron disease. As such, and for simplicity, various embodiments of the systems and methods described herein may be referred to as ‘assessment systems’ or ‘assessment methods’; however, it will be appreciated that various embodiments may relate to a system or method for various other applications, non-limiting examples of which may include lie detection, or oculomotor stimulation for, for example, brain injury or post-surgical rehabilitations, or cognitive training, without departing from the general scope or nature of the disclosure. Accordingly, it will be appreciated that an ‘oculomotor assessment’, an ‘assessment’, a ‘cognitive assessment’, or a ‘visual test’ or ‘visual assessment’, which may be referred to interchangeably herein, may additionally or alternatively relate to the monitoring of a user response to stimuli presented in accordance with an oculomotor exercise, training regime, or the like.

In accordance with various embodiments, various eye tracking systems or methods currently or as yet to be known in the art may be employed in within an HMD to record, monitor, and/or analyse, for instance, pupil size, position, movement, or the like, before, during, or after an oculomotor assessment. For instance, and without limitation, various eye tracking systems may relate to those employing visible or IR cameras to track pupils during vision-based, caloric, oculomotor, vestibular, or reaction time (OVRT) assessments. Similarly, and as will be further described below, an HMD may comprise one or more motion sensors, gyroscopes, accelerometers, or the like, to track user and/or head motion, or a relative motion, before, during, or after and assessment or exercise. Such data may be communicated to a practitioner to, for instance, inform assessments, therapy practices, or the like. That is, while some embodiments relate to direct processing of assessment data on-board an HMD or via a processing resource coupled therewith to filter, calculate, or infer various metrics related to oculomotor behaviour and/or function, various embodiments may additionally or alternatively relay data or inferred metrics for professional human analysis, depending on, for instance, the particular application, assessment, or metric at issue.

In accordance with various embodiments, a visual stimulus may be presented within an assessment system such that it may be perceived by a user as being disposed at a one or more designated locations, or moving in up to three dimensions, in accordance with various oculomotor assessments. As will be further described below, a stimulus may be provided in various forms, in accordance with various embodiments. For example, a stimulus or stimuli may comprise a linear, two-dimensional, or three-dimensional array of light sources that are independently addressable and distributed in physical space such that, when different sources are activated or presented as a target of focus for a user, the user attempts to focus in a different spatial regions within the HMD. In some embodiments, this may relate to a pixelated display screen in which individual pixels or groups thereof may be addressed and/or activated to provide a stationary or perceivably moving stimulus in accordance with a designated assessment. For example, a pixelated display screen may activate pixel groups as a stimulus to draw the user's gaze to a particular location in the plane of the screen, whereby the stimulus is digitally moved in the plane of the screen in accordance with an assessment. In accordance with yet other embodiments, such a screen may be further displaced along an axis perpendicular to the plane of the screen to further displace the stimulus in a third dimension. In such embodiments where a stimulus that is physically moving or appears to be moving, the stimulus may also be referred to herein as a ‘dynamic stimulus’.

In accordance with some embodiments, an oculomotor stimulus that is mechanically displaceable is provided by the HMD. As is further described below, such a mechanically displaceable stimulus may be presented and/or displaced by an actuation mechanism in accordance with an oculomotor assessment. In some such embodiments, the actuation mechanism, which may be manually or digitally operated, may displace the mechanically displaceable stimulus towards or away from the user while pupil and/or eye tracking monitors an oculomotor response thereto, so to enable, for instance, a vergence response assessment and/or an accommodation assessment.

The following description relates to the use of an HMD applied for the assessment of cognitive function or a cognitive disorder through assessment of the oculomotor system of a user. However, it will be appreciated that such applications are provided for exemplary purposes, only, and that the systems and methods described forthwith may be readily applied in, for instance, therapeutic and/or training applications, without departing from the general scope and nature of the disclosure.

In accordance with one embodiment, a head-mountable device is provided to assess a wearer's vestibular system, which comprises a set of organs and complex neural pathways that impart information related to a person's proprioception and equilibrium to permit appropriate compensatory reflexive movements. Indeed, the vestibulo-ocular reflex aims to achieve fixation and prevent image slippage. Various eye movement systems can be assessed, such, for example, saccadic, smooth pursuit, fixation, vergence, vestibulo-ocular and optokinetic. Insight abnormal behavior in any of the latter systems may provide valuable information on vestibular and oculomotor disorders. Eye tracking technology aided with tests that target any or each of these systems may permit insightful information on gaze stabilization. In one such embodiment, the device is operated as a video-nystagmograph (VNG), in which a computerized system applies the principle of recording eye movements using infrared-based (IR) camera sensors within a head-mounted device which obtains estimate measurements and analyzes eye movements using a computer software. In one embodiment, this is accomplished by displaying a stimulus for both eyes together on one display and each eye is recorded independently with a camera and infrared setup. Such 2D stimulus and response is combined with the selective deployment of depth-displaceable visual stimulus, as further detailed below, to provide a full suite of assessment capabilities.

In general, the device can be operated as a portable video-nystagmograph (VNG). The head-mounted device is designed to immerse the patient within a controlled environment while recording and estimating the eye movements. Tests are performed by the practitioners through a laptop, for example, provided with the device. Examples of testing capabilities that can be performed by such embodiments to permit the evaluation of pupillary responses, gaze-estimation and comfort/discomfort levels and thresholds, may include, but are not limited to: Predictive Horizontal Saccades; Predictive Horizontal Smooth Pursuit; Spontaneous and Gaze-evoked Nystagmus; Optokinetic Nystagmus; Subjective Visual Vertical Alignment; Positioning and Positional Nystagmus; Vergence; and/or Caloric Nystagmus.

As introduced above, and as will be described below with reference to the Figures, various embodiments as described herein relate to the provision of a head-mountable device having a mechanically displaceable oculomotor stimulus. For example, and without limitation, various embodiments relate to an HMD comprising an actuation mechanism operable to translate a stimulus along a track, groove, belt, or like structure towards or away from the user's face while the user's eyes are monitored, thereby performing, for instance, a vergence response, convergence insufficiency, and/or accommodation assessment. Additionally, or alternatively, an actuation mechanism may present, displace, or otherwise position a mechanically displaceable oculomotor stimulus such that the stimulus may be translated in a plane in two dimensions (e.g. up/down and towards/away from the user's eyes, in a plane normal to the user's gaze direction and at a designated distance from the user's eyes, or the like), or again in three dimensions to perform similar or alternative assessments.

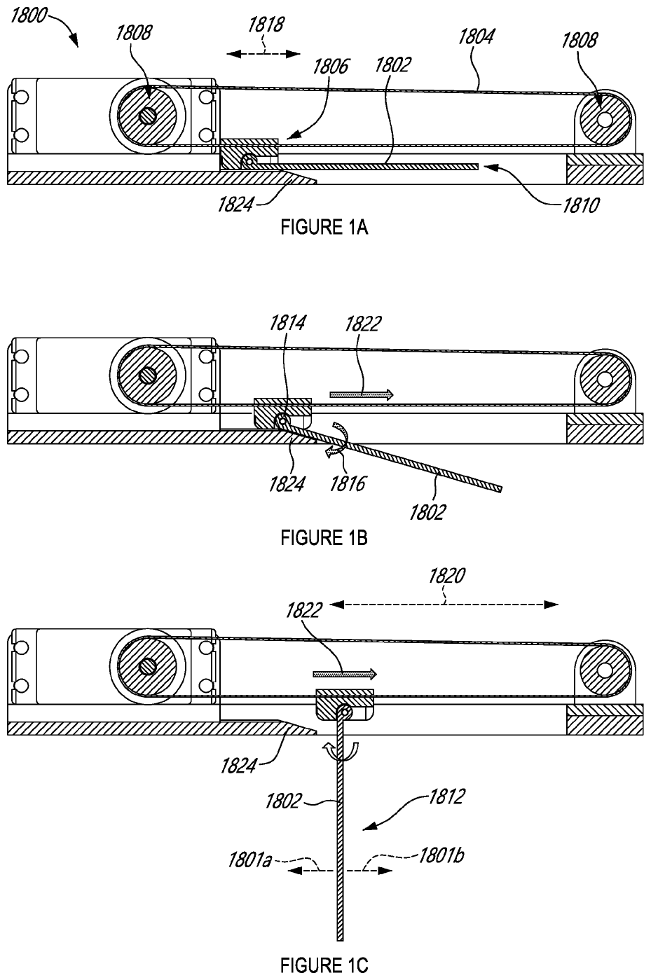

For example, and in accordance with one non-limiting embodiment, FIGS. 1A to 1C schematically illustrate one exemplary configuration of a head-mountable device (HMD) 1800 for performing an oculomotor assessment of a user. In this example, the HMD 1800 comprises a mechanically displaceable oculomotor stimulus 1802 that is displaceable via an actuation mechanism such that the stimulus 1802 is presentable to the user and may be displaced towards 1801a or away from 1801b the user's eyes. In the cross-sectional schematics of FIGS. 1A to 1C, the actuation mechanism is partially shown as a cable or belt 1804 to which the stimulus 1802 is coupled via a coupling means, in this case a clamp 1806 or like mechanism fixing the stimulus 1802 to the cable 1804 such that displacement of the stimulus 1802, cable 1804, and/or coupling means 1806 by a force initiates a corresponding motion of components coupled therewith. In this example, the cable 1804 is further configured about a pulley system 1808 such that the cable 1804 guides motion of the coupling means 1806 and stimulus 1804 coupled therewith along a defined path towards 1801a or away from 1801b the user. However, it will be appreciated that similar embodiments may employ alternative actuation mechanisms. For example, an actuation mechanism may comprise a threaded rod or like component such that a stimulus 1802 coupled therewith (e.g. in/via threading of the rod) is displaced towards or away from the user upon rotation (e.g. manual or electronically actuated rotation) of the threaded rod. Other such mechanisms, such as that employing a set of cogs, gears or the like, will be described below with reference to other illustrative embodiments.

With continued reference to FIGS. 1A to 1C, an HMD 1800 may be configured to provide a plurality of stimulus 1802 and/or device 1800 configurations. For example, FIG. 18A schematically illustrates the HMD 1800 having the mechanically displaceable oculomotor stimulus 1802 in a retracted configuration 1810, while FIG. 18C schematically illustrates the stimulus 1802 being configured in an extended or deployed configuration 1812. The provision of such configurations 1810 and 1812 may be beneficial to, for instance, present and displace the mechanically displaceable oculomotor stimulus 1802 in an extended configuration 1812 for performing an assessment employing the same, while otherwise removing the stimulus 1802 from the user's field of view, for instance to perform a complementary oculomotor assessment employing an alternative oculomotor stimulus.

For example, and without limitation, various embodiments relate to an HMD 1800 further comprising a display screen (not shown in FIGS. 1A to 1C) disposed at a designated or variable distance from the eyes of the user when the HMD 1800 is mounted to the user's face. The display screen may be operable to render a complementary two-dimensional oculomotor stimulus in accordance with a complementary oculomotor assessment, such as by activating or rendering designated pixels of the display screen to present a complementary stimulus to perform a saccade or pursuit assessment at the designated distance, as described above. During performance of such a complementary assessment, it may be preferred that the mechanically displaceable oculomotor stimulus 1802 be removed from the user's view and configured in the retracted configuration 1810. Upon performance of, for instance, a vergence response assessment, a convergence insufficiency assessment, an accommodation assessment, or the like, which may employ the mechanically displaceable oculomotor stimulus 1802, the HMD 1800 may be configured with the mechanically displaceable oculomotor stimulus 1802 in the extended configuration 1812 for presentation and/or displacement relative to the user.

In accordance with various embodiments, an HMD 1800 may comprise a mechanically displacecable oculomotor stimulus 1802 that is pivotably coupled with an actuation mechanism to provide the stimulus 1802 in a retracted configuration 1810 or an extended configuration 1812. For example, in the exemplary embodiment of FIGS. 1A to 1C, the stimulus 1802 is coupled with the coupling mechanism 1806 via a pivot joint 1814 about which the stimulus 1802 may rotate 1816. In this case, the stimulus 1802 configuration (i.e. retracted configuration 1810 or extended configuration 1812) is determined by its displacement relative to the user. That is, when the stimulus 1802 is positioned in a retaining position 1818 via, for instance, displacement from the actuation mechanism to, in this example, an extremum of its positional range, in this case to the left or near the user in FIG. 1A, the stimulus 1802 assumes a retracted configuration 1810.

As the stimulus 1802 is displaced away from 1822 from the user to a stimulus presentation position 1820, or towards the right in FIGS. 1B to 1C, via, for instance, the actuation mechanism, the stimulus adopts the extended configuration 1812.

It will be appreciated that a stimulus may be configured in accordance with various mechanisms and/or forces, in accordance with various embodiments. For example, in embodiments in which an actuation mechanism comprises an electric motor or like digitally manipulatable means, the stimulus may be actively and/or electronically configured in any one of a plurality of configurations by the actuation mechanism. Alternatively, a stimulus may be configured or reconfigured using any of various manual means, such as via a manual switch or lever, and/or may be retained in a retracted or extended configuration by various passive mechanisms. In the exemplary embodiment of FIGS. 1A to 1C, the mechanically displaceable oculomotor stimulus is retained in a retracted configuration 1810 via a retaining structure 1824. In this case, as the stimulus has a displacement relative to the user corresponding with a stimulus presentation position 1820 (e.g. its configuration is unimpeded by the retaining structure 1824), the force of gravity draws the stimulus into the extended configuration 1812. However, it will be appreciated that various additional or alternative mechanisms may similarly configure and/or support the stimulus in a configuration suitable for presentation. For example, one or more springs and/or a supporting mechanism, such as a grooved plate or other structure, may configure and/or support the stimulus 1802 in an extended configuration.

In the exemplary embodiment of FIGS. 1A to 1C, the retaining structure 1824 provides a means of reconfiguring and supporting the stimulus in the retracted configuration 1810. From the extended configuration 1812, as the actuation mechanism draws the stimulus 1802 towards the stimulus retaining position 1818, the retaining structure 1824 deflects the stimulus 1802 upwards about the pivotable coupling 1814, and thus outside of the field of view of a user having the HMD 1800 mounted to their face. The stimulus 1802 is then retained in the retracted configuration 1810 while the actuation mechanism maintains the stimulus 1802 in the stimulus retaining position 1818. As with the extended configuration 1812, it will be appreciated that various additional or alternative retention means or mechanisms may similarly support the mechanically displaceable oculomotor stimulus in the retracted configuration 1806, in accordance with various embodiments.

In the exemplary embodiment of FIGS. 1A to 1C, the mechanically displaceable oculomotor stimulus 1802 comprises a fixation member (e.g. stick) that may be mechanically displaced towards or away from the user to perform, for instance, a vergence and/or accommodation assessment. In some embodiments, the stimulus 1802 serves as a target of focus for the user during the assessment, wherein they may be asked, for instance, to provide subjective feedback on their comfort level and/or ability to perceive the stimulus at one or more, or indeed, varying, distances from their eyes. In accordance with some embodiments, such a stimulus 1802 may additionally comprise one or more indicia thereon, for instance to further serve as a target for the focus of the user. For example, and without limitation, one embodiment relates to a stimulus comprising a fixation stick 1802 in turn having depicted thereon (e.g. as an image, an applied label, a sticker, or the like) one or more indicia, such as a Snellen chart, one or more letters (e.g. the letter “E” stamped, embossed, or otherwise printed or labelled), numbers, or symbols, optionally of varying sizes, or the like, to facilitate and/or provide an assessment. For instance, the user may be asked to provide verbal feedback on a blurriness of a letter presented via the stimulus as the stimulus is made to approach the user's eyes via an actuation mechanism, or a comfort level when focused on the stimulus or indicia thereon. Similarly, a user may be asked to report when the stimulus or indicia is perceived in double. The skilled artisan will appreciate that various similar or other oculomotor and/or cognitive assessments may be facilitated by an HMD 1800 comprising a mechanically displaceable oculomotor stimulus 1802, without departing from the general scope and nature of the disclosure.

While the stimulus 1802 of the HMD 1800 comprises a fixation stick 1802, it will be appreciated that other forms of a mechanically displaceable oculomotor stimulus may be provided, in accordance with other embodiments. For example, and without limitation, an HMD 1800 may comprise an actuation mechanism operable to displace an anchoring or like component (e.g. the coupling mechanism 1806) to which a Snellen chart, card, or other like stimulus may be provided (e.g. in a reversible fashion) and displaced via the actuation mechanism.

In addition, or as an alternative, to subjective oculomotor and/or cognitive assessments, various embodiments relate to the acquisition, processing, and/or communication of objective oculomotor metrics and/or quantitative data regarding an assessment. For example, various embodiments of an HMD 1800 comprise a pupil or eye tracking device monitoring the user's eyes as they perform an assessment and/or complementary assessment. As described above, such assessments may relate to the reporting of quantitative metrics associated with oculomotor behaviour, such as an interpupillary distance, gaze direction, number and/or degree of microsaccades, gaze accuracy and/or stability, or the like, depending on, for instance, the assessment being performed. As described above, such metrics may relate to a risk of a potential health condition, such as a concussion or other form of TBI, which may be reported or otherwise indicated to a practitioner, or may relate to raw, filtered, and/or processes oculomotor data, for instance for presentation via a GUI associated with an operator device, and/or storage for future consultation by the practitioner. The skilled artisan will appreciate that various components, devices, or systems related to eye or pupil tracking, such as one or more sensors or cameras, pupil and/or corneal illumination sources, computer resources, or the like, may thus be incorporated without an HMD to this end.

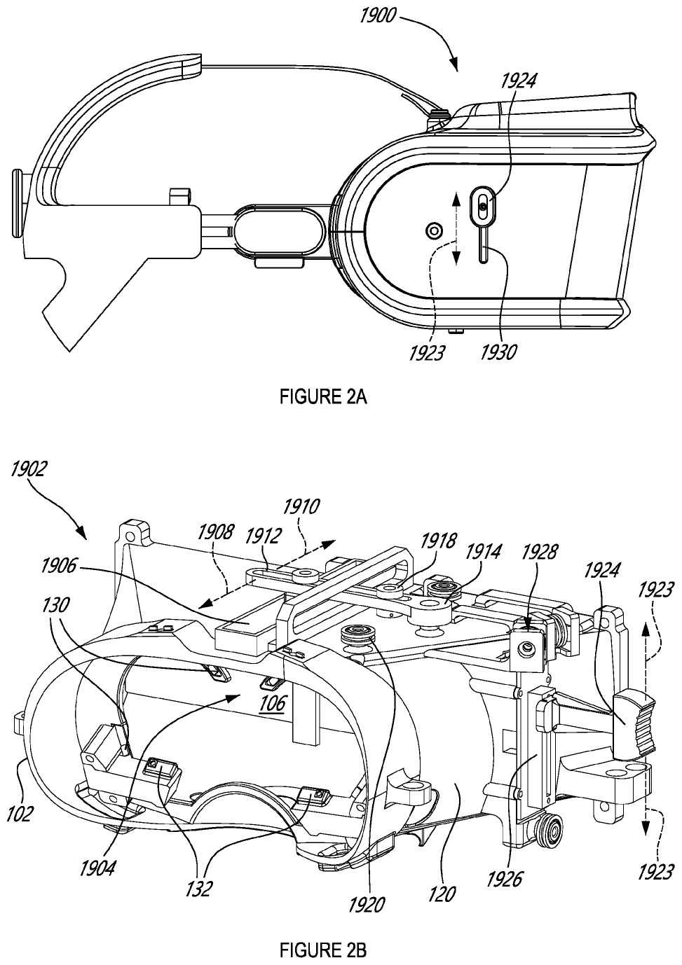

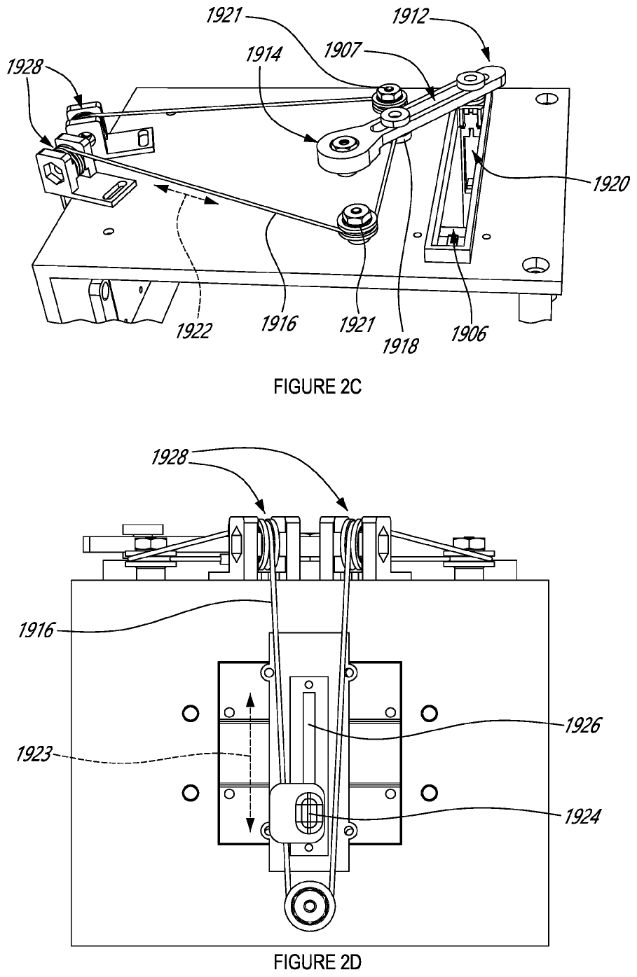

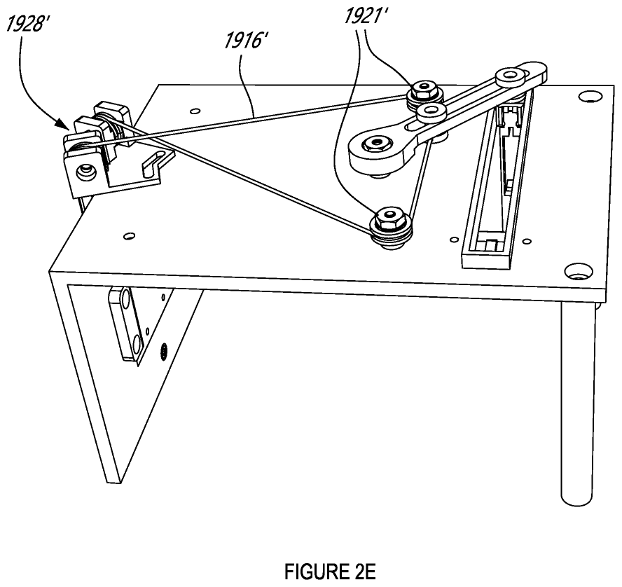

With reference now to FIGS. 2A and 2B, various embodiments relate to a head-mountable device 1900 comprising a mechanically displaceable oculomotor stimulus that is displaceable using a manual actuation mechanism, such as that described above with reference to FIGS. 1A to 1C. For example, FIG. 2A is a schematic showing a side view of a head-mountable device 1900 having an exemplary manual actuation mechanism, and FIG. 2B is top rear right-side view of various inner components of the exemplary manual actuation mechanism of the device 1900 of FIG. 2A. In this example, the device 1900 comprises a head-mountable housing 1902 that, when mounted to the user's face, defines an internal visual stimulation chambre 1904 therein for presenting an oculomotor stimulus (e.g. a mechanically displaceable oculomotor stimulus, a complementary stimulus, such as one for performing saccade or pursuit assessments and rendered using a display screen, or the like), such as the head-mounted device described above. While FIG. 2B schematically illustrates some of the various internal components of an exemplary manual actuation mechanism, FIGS. 2C and 2D further illustrate the interconnectivity of various mechanism components, as well as additional and/or complementary components to those schematically shown in FIG. 2B.

In these non-limiting examples, a mechanically displaceable stimulus 1920 is configured to protrude within the housing 1902 through an aperture, gap, or slot 1906 therein to be visible to a user within the visual chambre 1904 of the housing 1902. The stimulus is operable to be displaced towards 1908 or away from 1910 the user's eyes via a corresponding displacement of a pivoting arm 1912 engaged therewith. In this example, the stimulus 1920 may protrude through both the housing slot 1906 and slot 1907 in the pivoting arm 1912 such that, as the pivoting arm 1912 pivots about a pivot means 1914 anchored to the housing 1902, the stimulus 1920 is operable to translate through both the housing slot 1906 and slot 1907 in the pivoting arm 1912 to be displaced towards 1908 or away from 1910 the user's eyes.

Rotation of the pivoting arm 1912 about the pivot means 1914 may be achieved via coupling with a cable 1916 or other actuation means (e.g. a belt, chain, or the like) by way of a coupling means 1918, such as a clamp or friction fitting. As the cable 1916 is pulled 1922 under tension, for instance about a pulley or disk system 1921, the coupling means 1918 initiates rotation of the pivoting arm 1912 about the pivot 1914, wherein, through engagement with both slots 1906 and 1907, the stimulus 1920 is displaced towards or away from the user. It will be appreciated that the particular configuration and/or geometry of various components (e.g. pivots points, relatively positions thereof, the length or relative length of the pivot arm, or the like) may define varying and/or different degrees of translation of the stimulus 1920 for a given displacement 1922 of the cable 1916 or other like actuation means. Also, as illustrated in FIG. 2E, an alternate configuration of pulley system 1928′ can actuate a similar motion of the oculomotor stimulus while crossing the cables 1916′ in reaching pulleys 1921′. Other such variations and equivalents will be readily appreciated to fall within the general scope and nature of the present disclosure.

With continued reference to FIGS. 2A to 2D, in this example, tension for displacing the pivot arm 1912, thus producing a corresponding translation of the mechanically displaceable oculomotor stimulus 1920 within the chambre 1904, may be provided by a corresponding displacement of a manually manipulable user-interfacing tension producing mechanism 1924. For simplicity, such a mechanism may also be referred to herein as a ‘lever’ 1924, although it will be appreciated that various configurations do not necessarily relate to a lever as a means of amplifying a force or displacement applied thereto, as may be the case of the mechanism employed a fulcrum or like component, although such aspects are similarly contemplated within the general scope and nature of the disclosure. Similarly, a lever 1924 may additionally or alternatively comprise a knob, dial, button, or other like mechanism that may enable a manual displacement of a mechanically displaceable oculomotor stimulus.

In the exemplary embodiment of FIGS. 2A to 2D, the lever 1924 is disposed on the side of the head-mountable device 1900 and housing 1902. That is, while the pivot arm 1912 disposed atop the device 1900 and housing 1902 rotates in a plane generally characterised by the top surface of the device 1900 as a result of the forces transmitted from the cable 1916 in that same plane, the force generating motion is applied in a direction generally perpendicular thereto, in this case via manual manipulation of the lever 1924 the plane generally characterised by the side of the housing 1902 of the device 1900. In this case, the lever 1924 is coupled (e.g. clamped, of friction-fit) with the cable to transfer a force and/or motion thereof to the cable to initiate a corresponding motion of component atop the device. Accordingly, by manually displacing 1923 the lever 1924 vertically, the cable 1916 is translated or rotated vertically in the plane characterising the side of the device. The forces and motion of the cable are then redirected to a plane generally characterising the top surface of the device by a pulley system 1928, ultimately displacing the mechanically displaceable oculomotor stimulus in a direction towards or away from the user. In this non-limiting example, lever motion 1923 is generally guided by a track 1926 or like feature of the housing 1902, although the skilled artisan will appreciate that various other mechanisms and/or components may be similarly applied to guide and/or support manual manipulation of the lever 1924, such as a groove or slot 1930 in a device casing.

Such a configuration in which a stimulus 1920 motion towards 1908 or away from 1910 the user is generated from a manual displacement 1923 in a different plane or direction may be beneficial for a number of applications, in accordance with various embodiments. For example, for embodiments in which actuation of the stimulus 1920 is manually achieved, displacing a lever 1924 or like mechanism vertically may allow for more natural and stable movement. For example, some embodiments relate to an HMD 1900 that is to be held by the user of the device during use. The body configuration of the user when required to hold and operate the device may naturally allow for smooth manipulation of the lever 1924 with their hands or fingers in a vertical direction. Conversely, manipulation of the lever mechanism in the forward and backwards directions may reduce the stability of the device and/or user when the device is in use, thereby reducing performance of an assessment and the quality of any results obtained therefrom, as well as increasing the risk of exacerbating a potential injury, such as if the user is suspected of having a neck injury and/or concussion. However, it will be appreciated other configurations may be adopted, depending on, for instance, expected use cases and/or applications. For example, some embodiments relate to an HMD that is to be supported by a practitioner, for instance in field scenarios in which there is a high risk of head and neck injuries. In such cases, it may be preferred that an actuation mechanism be displaceable exclusively in a direction towards and away from the user's eyes, or in accordance with an alternative configuration of forces and component displacements.

In accordance with different embodiments, it may be preferred that a mechanically displaceable oculomotor stimulus be actuated via either a manual actuation mechanism, as described above with respect to FIGS. 2A to 2D, or an electric or automated actuation mechanism. For example, it may be preferred for some applications that displacement of a stimulus be fully automated and/or digitally controlled. Some embodiments, for instance, comprise a digital and/or electric motor (e.g. a stepper motor, a server motor, or the like) in communication with a digital data processor such that displacement of the stimulus may be automatically or remotely controlled thereby, such as by a practitioner operating a graphical user interface (GUI) via an operator device. For instance, one embodiment relates to operator control of an oculomotor assessment via an operator application digitally executable on a distinct operator device having an operator display screen and a communication interface to the HMD. The operator application may comprise digitally executable instructions to render the GUI on the digital display screen, and receive as input therefrom manual digital control of the mechanically displaceable oculomotor stimulus such that a stimulus displacement thereof corresponds with a manual displacement entered via the GUI. In accordance with a similar embodiment, a digital actuation mechanism may automatically execute a pre-defined ocular assessment, such as by executing digital instructions to initiate and control the automatic translation of the stimulus in accordance with a designated oculomotor and/or cognitive assessment defined through digital machine-executable instructions defining a range of stimulus positions and translation rate therebetween.

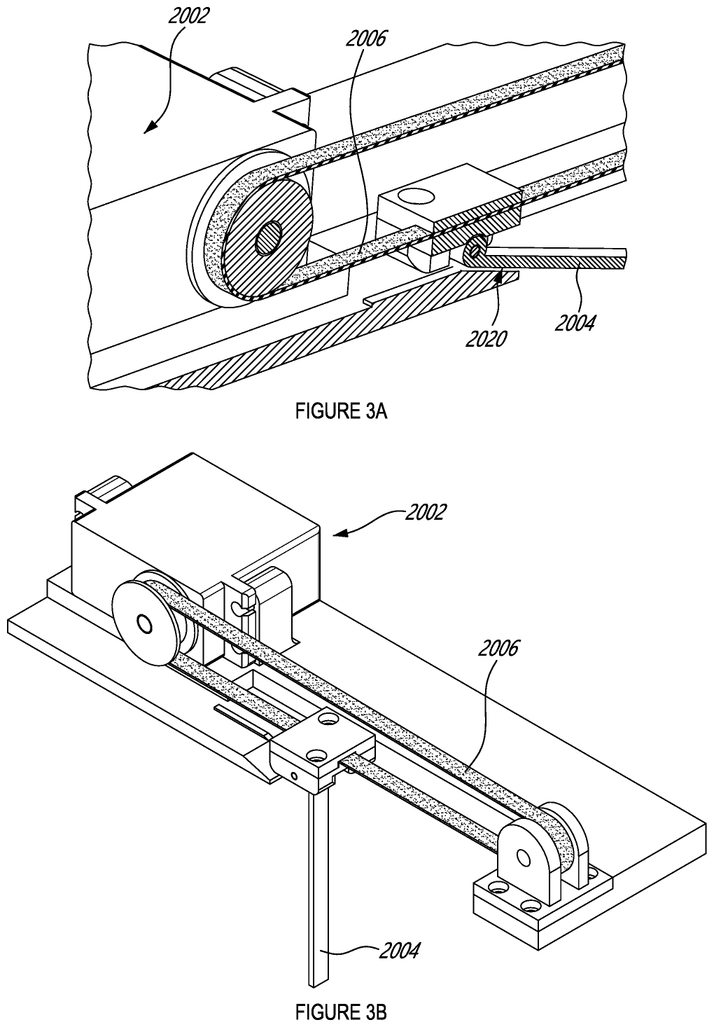

In accordance with one such embodiment, FIGS. 3A and 3B are schematics of an HMD comprising a digitally controlled actuation mechanism. In this example, a casing 2002 houses a digital motor (e.g. a stepper motor, a servo motor, or the like), and may further house any power sources, electronics, digital storage media, or the like, required to provide a force or motion to displace an oculomotor stimulus 2004. In this case, the digital actuation mechanism in housing 2002 actuates a conveyor belt 2006 which in turn displaces the oculomotor stimulus 2004. Similar to the device of FIGS. 1A to 1C, the device of FIGS. 20A and 20B comprises a retaining structure to retain the stimulus 2004 in a retracted position upon the actuation mechanism displacing the stimulus 2004 to a retaining position.

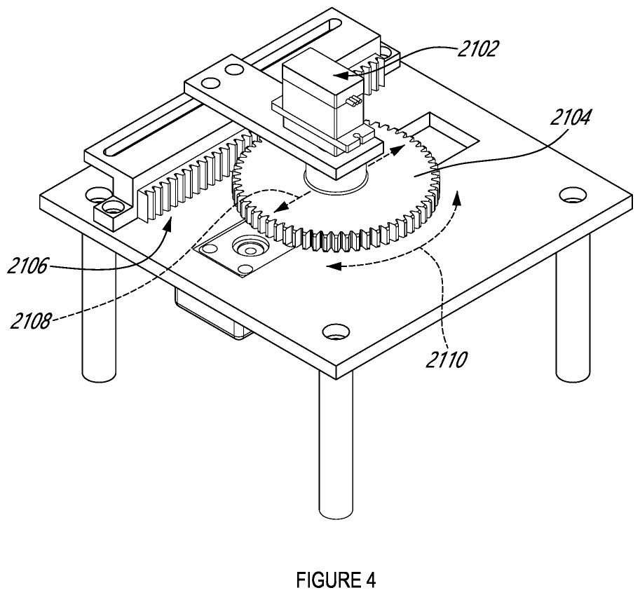

In accordance with yet another embodiment, an alternative configuration for a digital actuation mechanism is schematically shown in FIG. 4. In this example, a digital motor 2102 is configured to rotate a gear 2104 or cog 2104 operatively engaged with a generally linear structure 2106 oriented generally along the path 2108 of displacement of the oculomotor stimulus (not shown in FIG. 4), wherein the structure 2106 comprises grooves or teeth complementary to those of the cog 2104. Upon rotation 2110 of the cog 2104 by the digital motor 2102, engagement of the cog 2104 with the grooves on the linear structure 2106 initiates linear translation 2108 of the cog 2104, mechanically displacing an oculomotor stimulus coupled thereto in a direction towards or away from the user's eyes in accordance with an oculomotor or cognitive impairment assessment.

In accordance with yet other embodiments, an actuation mechanism for displacing a mechanically displaceable oculomotor stimulus may comprise a pneumatic and/or hydraulic device, or like mechanism. In yet another embodiment, an actuation mechanism may comprise a muscle wire, such as a nitinol-based wire.

While some embodiments contemplated herein indeed relate to a digital actuation mechanism, various applications may benefit from a manual actuation mechanism. For example, and in accordance with various embodiments, a manual actuation mechanism may be of benefit for reducing vibration or other motion that may be detrimental or even harmful to a user of the device, such as one suspected of a brain or neck injury. Accordingly, various embodiments may relate to the use of exclusively manual actuation means. It will be appreciated that such embodiments may offer further benefit of having reduced weight, complexity, and/or cost as compared to digitally actuated devices.

In accordance with embodiments comprising a digital actuation mechanism, it will be appreciated that a stimulus displacement or position relative to the user may be readily known, monitored, and/or recorded via the digital actuation mechanism and/or associated digital application. For instance, a stepper motor may displace a stimulus in accordance with a known step size, wherein the number of steps taken to displace the stimulus is digitally monitored and recorded (and optionally displayed, for instance via a GUI associated with an operator device). It will be appreciated that such steps or other displacements may be calibrated against a displacement of the mechanical stimulus. Additionally, or alternatively, a stimulus position or displacement may be monitored by additional or alternative means. For example, and without limitation, a potentiometer or variable resistor may be coupled to one or more components of a device, wherein the resistance of the potentiometer is a function of the position of the mechanically displaceable oculomotor stimulus, or a component coupled therewith. The resistance may thus be measured to extract a position of the oculomotor stimulus during assessment. It will be appreciated that various calibration processes may first be employed to accurately determine a stimulus displacement from resistance measurements. For example, resistance measurements of a potentiometer may be calibrated to correspond to a known stimulus displacement with sub-millimeter precision and/or accuracy, in accordance with some embodiments.

For example, and in accordance with one embodiment, a potentiometer may be coupled with the lever 1924 of FIGS. 2A to 2D. For instance, the lever support 1926 of FIG. 2D defines a variable resistor having a resistance in turn defined by the vertical position 1923 of the lever 1924, which may be monitored as the lever (and thus the oculomotor stimulus coupled thereto) is displaced during an assessment. It will again be appreciated that such resistance measurements may be calibrated for a particular device and/or device configuration. Similarly, it will be appreciated that a potentiometer or like device may be employed elsewhere in the device. For instance, a potentiometer may be installed on a top surface of the housing 1902 such that the position of coupling means 1918 defines a resistance measured from the potentiometer, which may in turn be monitored and recorded, and again may correspond with a displacement or position of the oculomotor stimulus relative to the eyes of a user of the device.

It will be appreciated that the length of a variable resistor or potentiometer need not necessarily correspond to an extent of the range of motion of the oculomotor stimulus.

For example, various embodiments relate to a range of positions accessible to a stimulus spanning 10 cm, whereas a range of contact positions of a coupled potentiometer may span, for instance, 2 cm, or 5 cm. That is, an extent to which a device component may interact with a potentiometer or like mechanism may not correspond directly to a range of motion of a stimulus, as a device geometry or components of the device may enable an amplification in the range of accessible displacements, as will be appreciated by the skilled artisan.

In accordance with yet other embodiments, a stimulus position or displacement may be directly measured during an assessment. For example, a device or device housing may further comprise a laser or other depth measurement device configured to monitor a stimulus position over the course of an assessment, and communicate and/or record the same.

In accordance with various embodiments, an HMD comprising a mechanically displaceable oculomotor stimulus may further comprise one or more illumination sources so to enable and/or facilitate perception of the stimulus by the user. For example, while one embodiment relates to a head-mountable device that allows ambient light to illuminate a stimulus, other embodiments relate to the provision of a device housing that provides a visual stimulation chambre, which may optionally be configured to be immersive and comprise inner surfaces that dampen or otherwise reduce reflections, as described above.

Accordingly, and in accordance with various embodiments, an illumination source(s) may be provided within a device to illuminate a stimulus to facilitate an assessment. For example, and without limitation, one or more LEDs may be provided within an HMD to illuminate a stimulus, in accordance with various embodiments. It will be appreciated that such light sources may be disposed within the device depending on, for instance, a device configuration. For example, one embodiment of an HMD comprises two LEDs, each mounted near or proximate to a face-mounting portion of the device and directed away from the user's eyes. In accordance with some embodiments, such illumination sources may further comprise or be coupled with one or more diffusers or like mechanisms to provide a suitable or healthy lighting condition in which to perform an assessment. In accordance with one embodiment, an illumination source may comprise a display screen or portion thereof disposed within the HMD, such as a wide screen display configured to provide a complementary stimulus for performing a complementary assessment (e.g. a saccade assessment).

In accordance with some embodiments, a head-mountable device may be configured to provide a range of motion or displacement of a mechanically displaceable oculomotor stimulus corresponding to a distance of between 5 cm and 20 cm. In accordance with one embodiment, the range of positions accessible to a stimulus for performing an assessment (e.g. the range of presentation positions 1820 of FIG. 1C) spans approximately 10 cm.

In accordance with different embodiments, a head-mountable device may be configured such that a mechanically displaceable oculomotor stimulus thereof may be displaced via an actuation mechanism manually operated by a user of the device, thereby allowing the user control over stimulus displacement in view of, for instance, comfort levels during an assessment. In accordance with other embodiments, the device may be configured such that stimulus position may be manually controlled via a practitioner or other external party. In accordance with yet other embodiments, a stimulus displacement may be manually and digitally controlled, for instance via a GUI associated with an external computing device. In accordance with yet other embodiments, a stimulus displacement may be automatically actuated in accordance with a digitally defined assessment stored in digital media and executable by a digital data processor.

It will be appreciated that various embodiments of a head-mountable device comprising a mechanically displaceable oculomotor stimulus may additionally comprise any one or more aspects of a head-mountable device described herein. For example, an HMD comprising a mechanically displaceable oculomotor stimulus may further comprise a widescreen display for performing 2D complementary assessments, and may comprise an immersive housing. Similarly, an HMD may comprise an external assessment status or result indicator externally viewable to a bystander of an assessment.

Moreover, while various embodiments relate to the performance of subjective oculomotor assessments, various embodiments may comprise eye or pupil tracking devices and/or functionality. For example, even in subjective assessments, user pupil positions may be recorded for real-time or subsequent analysis by a physician or other practitioner. In accordance with yet other embodiments, such pupil or eye tracking data may be automatically analysed for, for instance, a risk of a condition, in real-time, and a risk of a potential condition may be automatically reported to a practitioner. Accordingly, various embodiments relate to the provision of a device, or a method employing a device, that enables fully automated assessments of oculomotor and/or cognitive behaviour.

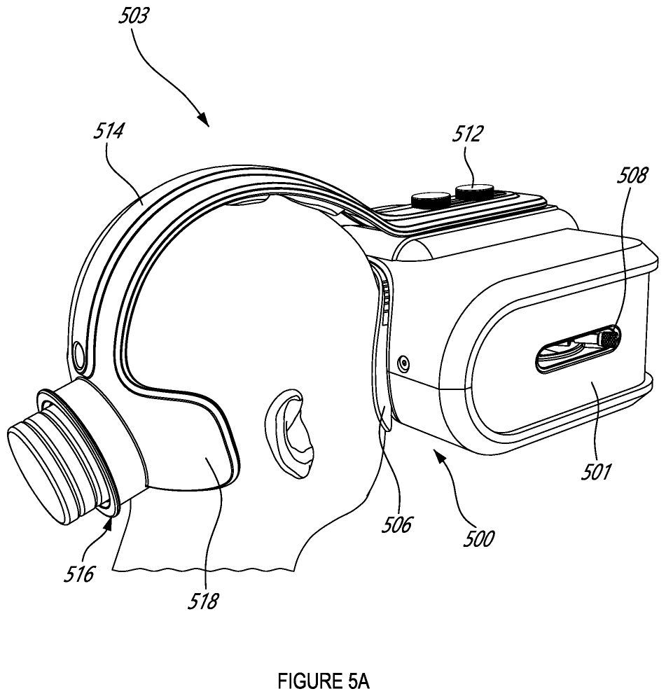

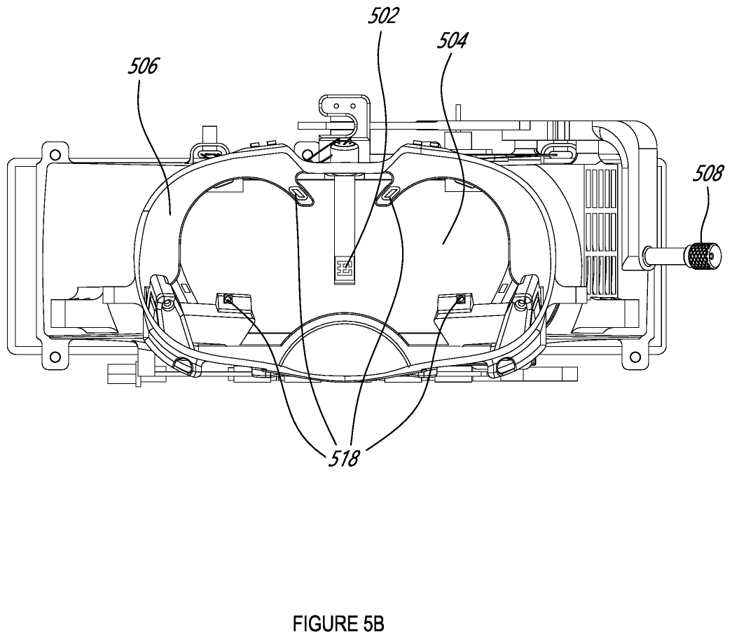

With reference to FIGS. 5A to 9B, and in accordance with yet another embodiment, a head-mountable assessment device 500 will now be described. In this embodiment, the device 500 again comprises a head-mountable housing 501 that, when mounted against a user's face using a corresponding head harness, strap or in this case, a spring-biased head-mountable frame 503, defines an internal visual stimulation chamber having a stimulation mechanism therein. In embodiments related to oculomotor assessments, such a stimulation mechanism may comprise, for example, a mechanically displaceable oculomotor stimulus 502 and a display screen 504, for example, which may include a light field display or the like. Accordingly, various embodiments relate to a head-mountable housing 501 comprising similar attributes to those described above with respect to other HMD embodiments, such as an immersive and/or dark internal chamber, a face-resting portion 506, a lever or like actuator 508 to manually adjust a mechanical stimulation mechanism, an external visual indicator, or the like. Various embodiments will further be understood to comprise various electrical and structural components necessary for operation of a stimulation mechanism, such as actuators and required electrical and/or optical circuitry, as illustratively described in greater detail below.

In the illustrate embodiment, the head-mountable frame 503 comprises a support frame in turn comprising a housing coupling portion 512 configured to retain the head-mountable housing 501 against the user's face at a forward region of the support frame, and a counterweight portion 516 configured to support a counterbalancing mass at a rearward region of the support frame. A spanning member 514 couples the housing coupling portion 512 and the counterweight portion 516, and is generally configured to contour and/or conform to the head and/or cranium of the user, thereby distributing device weight thereacross (e.g. front-to-back, with lateral stabilisation provided through spanning member width, one or more head-interfacing stabilisation structures, and/or head- interfacing padding).

Generally, the spanning member 514 may be arcuate and contour and/or conform to an upper region of the user's head and/or cranium, as schematically illustrated in FIG. 5A. In accordance with some embodiments, the spanning member 514 may further contour the back of the user's head, and, in some embodiments, further descend a portion of the user's neck (i.e. the nape of the neck) and supportively interface therewith (518). Accordingly, in some embodiments, the spanning member 514 may distribute the weight of the device when worn, and may, in accordance with some embodiments, dispose device mass or weight in/at a counterweight portion 516 that is lower than the head-mountable housing 501 when the device is worn (and optionally maintaining a counterbalancing weight close the user's head), thereby lowering the centre of gravity of the device when in use as compared to conventional systems. As illustrated by the illustrated embodiments, a counterweight location may be disposed or aligned, when the device is worn, somewhere toward the base of the skull, or lower toward or approaching the upper nape of the user's neck.

With particular reference to FIG. 5B, an internal view of the head-mountable housing is provided in which disposition of the mechanically displaceable visual stimulus 502 in its deployed configuration is observed as centered with the face resting portion 506 and deployed in front of a complementary 2D assessment display screen 504. In this particular view, a pupil tracking system is also illustrated consisting of right and left camera systems 518 (e.g. respective IR light source and camera systems for monitoring pupil characteristics and/or motion in the dark isolated environment of the stimulation chamber).

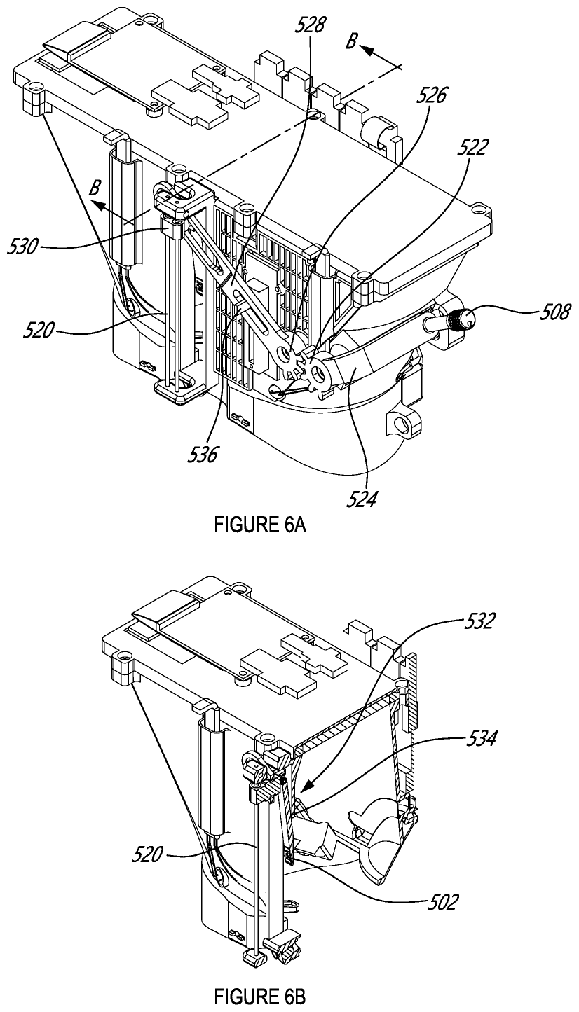

With particular reference now to FIGS. 6A to 9B, a manual actuation mechanism for the mechanically displaceable visual stimulus will now be described in greater detail. In general, the mechanical actuation mechanism translates a forward and backward rotation of the actuation lever 508 into a forward and backward linear translation of the visual stimulus 502 along a corresponding track 520. In particular, the lever 508 is rigidly coupled to a first spur gear 522 that, when rotated via spur gear arm 524, imparts a corresponding and opposite rotation of a second spur gear 526. This second spur gear comprises an extended arm 528 that mechanically engages a visual stimulus coupling 530 slidingly mounted to slide back and forth along the track 520 via a set of linear bearings in response to a sweeping rotation of said extended arm 528. The extended arm 528 also mechanically engages a potentiometer 632, or like component, that can translate a rotation of the extended arm 528 into an electrical signal representative of a position of the mechanically displaceable stimulus 502. Indeed, a calibrated potentiometer or like position sensing device can relay positional information such as retracted vs. deployed, and a deployed position once deployed along the track 520 to contribute to a testing or screening out of the device. This position or status can be relayed for display on a GUI or output provided to interface with the device, and tracked along with a user's oculomotor response thereto (or like vision-based responses) to output a screening or testing result or indicum.

In FIGS. 6A and 6B, the mechanically displaceable stimulus 502 is illustrated in its fully retracted position, with the coupling 530 in its rearmost position along track 520, mechanically retracting the stimulus member 502 against retaining structure 532 (i.e. rearward extending wedge portion 534 that urges the stimulus member 502 to pivot upward and retract up against and within retaining structure 532). Other mechanical retaining structures and mechanisms may also be considered, as will be readily appreciated by the skilled artisan.

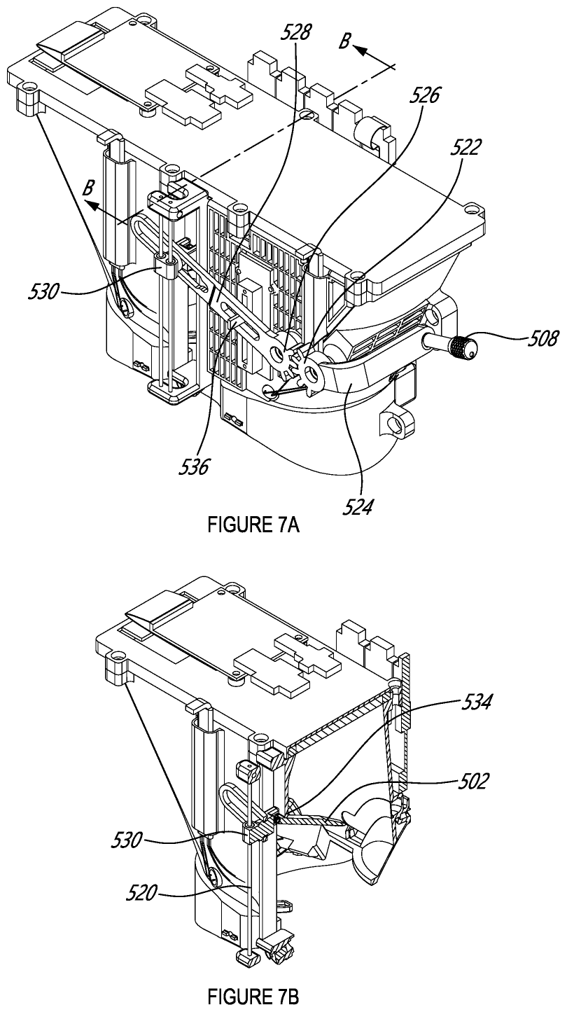

In FIGS. 7A and 7B, the mechanically displaceable stimulus 502 is illustrated in its partially deployed position, with the coupling 530 in an intermediate position along track 520 that coincides with the stimulus member 502 being mostly deployed about the tip of the wedge portion 534 (e.g. under action of gravity or via a spring bias, for example, provided by a torsion spring or the like engaged between the stimulus member 502 and coupling 530) such that it becomes visible to a wearer of the device.

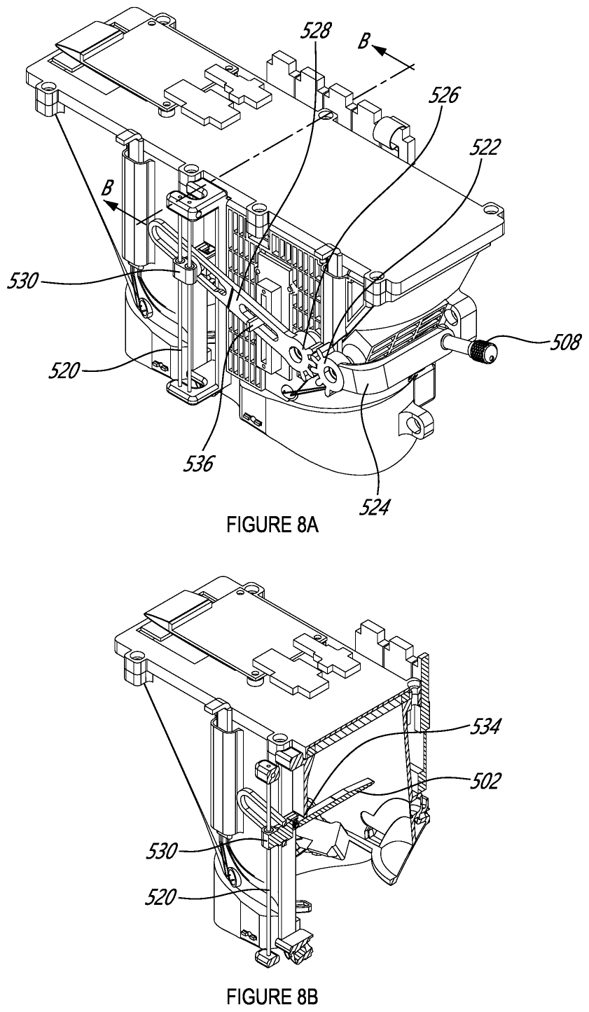

In FIGS. 8A and 8B, the mechanically displaceable stimulus 502 is illustrated in its rearmost fully deployed position, with the coupling 530 in an intermediate position along track 520 that coincides with the stimulus member 502 being fully deployed beyond the tip of the wedge portion 534, thus allowing the viewer to focus on the mechanically displaceable stimulus 502 in its rearmost stimulus position.

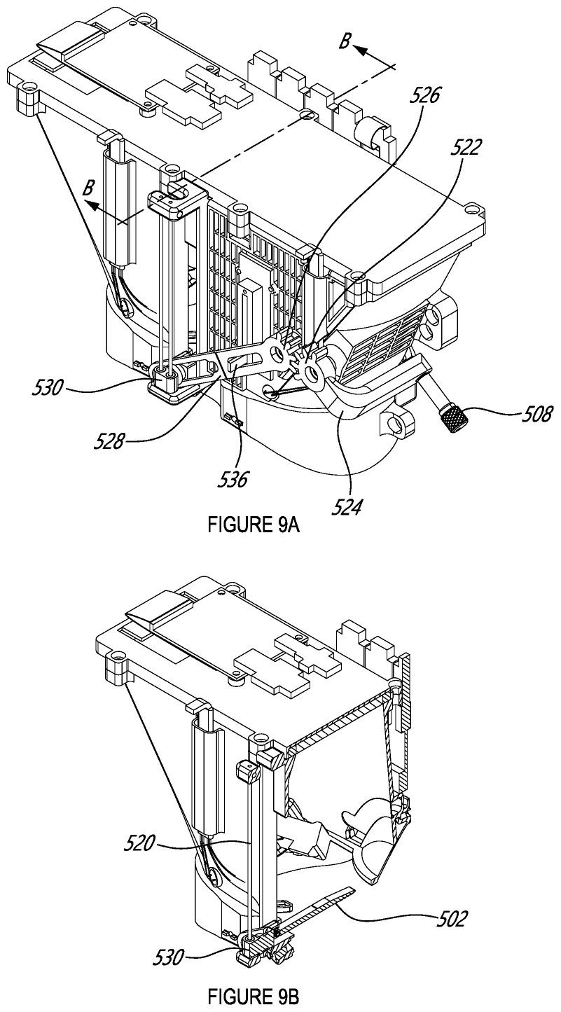

Finally, in FIGS. 9A and 9B, the mechanically displaceable stimulus 502 is illustrated in its foremost fully deployed position, with the coupling 530 in its foremost position along track 520 that coincides with the stimulus member 502 being positioned nearest to the viewer. Accordingly, the mechanically displaceable stimulus can be displaced fore and aft along the track 520 so to visually stimulate a wearer of the device while tracking a response thereto in correlation with a tracked position of the visual stimuli.

As noted above, and in accordance with one embodiment, a head-mountable device (HMD) operable to perform a cognitive impairment assessment and/or screening, make take various forms and enclose various complementary assessment tools and/or technologies. Different such examples are described and illustrated, for example, in Applicant's co-pending International Application No. PCT/US2022/013564, the entire content of which is incorporated herein by reference.

Returning to the example shown in FIGS. 2A and 2B, for simplicity, the device 1900 may comprise a face-resting portion 102 that is to be aligned and rested around the user's eyes on their face, and maintained in position via a head-fastening strap or harness 1904 that can be adjusted to secure the HMD 1900 to the user's head. Various head-mounting, weight-bearing and/or otherwise supportive mechanism, as described herein, may equally be considered herein, as such for example by the embodiment of FIG. 5A, as can various mechanical adjustments, interfaces and/or motion-related accommodations may be equally considered within the present context. Similarly, it will be appreciated that the HMD, face-resting portion, and/or harness may be equipped with various pads or physical structures for user comfort and/or isolation, and/or to provide additional functionality. For example, various embodiments relate to the face-resting portion comprising a padding or flexible component for user comfort and stability during use. In accordance with some embodiments, such a padding may optionally additionally or alternatively serve as a means of blocking external light during performance of one or more assessments, thereby improving user experience and/or the quality of assessments, which may otherwise be hindered by stray light entering the system that may distract the user or contribute to discomfort.

As noted above, the device may enable an assessment to be conducted via a visual interface within the HMD. In some exemplary embodiments, an external indicator, such as a colour-coded luminous indicator, is provided in a rearward facing configuration (or indeed, an indicator disposed on the HMD such that it is readily visible to an external non-user of the device) such that a test administrator, or anyone in the presence of the user during performance of the test, can visually obtain an immediate screening result while the user is wearing the device. For example, a red indicator may indicate that a test screening suggests the user is at a relatively high risk of exhibiting cognitive impairment, whereas a green indicator may indicate a lower likelihood of cognitive impairment. Other colour indicators may, for example, reflect that a screening test is underway (i.e. active screening), or again flash or change colours based on a current status or progression of the device. However, unlike a tradition head-mounted device where outputs are constrained to being rendered by the device's internal display, for instance within the context of a virtual reality device or the like, or again, wherein external outputs or results are exclusively made available via a communicatively linked device (e.g. tethered laptop or remote display), the device provides a local operator, administrator and/or assistant (e.g. nurse, doctor, family, friend, bystander, coach, technician, etc.), immediate indication of a screening status in a readily available configuration. Indeed, one assisting a user in the implementation of the test is likely to stand in front of them, thus facing a rearward extent of the device, and thus, conveniently positioned to acknowledge an output of the indicator. As will be appreciated by the skilled artisan, other types of indicators may be considered without departing from the general scope and nature of the present disclosure. Moreover, various embodiments relate to the use of such an indicator on various other HMD configurations. For instance, a light-field based HMD, and/or an HMD comprise a plurality of parts, as will be further described below, may similarly employ an outward-facing assessment status indicator, without departing from the general scope and nature of the disclosure.

The HMD may further comprise various additional external components, depending on the particular application at hand. For example, and without limitation, the HMD or harness may be equipped with speakers to provide a user of the device with audio content, such as assessment instruction, guidance, and/or feedback before, during, or after an assessment. Such a feature may be of benefit if, for instance, a practitioner is guiding an assessment remotely, or if such guidance is pre-recorded, or computer-generated in real-time in response to assessment progression and/or results. Speakers may further be of value in expanding the range of tests that may be performed with the HMD. For instance, while various embodiments relate to the presentation of visual stimuli, other assessments may additionally or alternatively relate to a volume sensitivity in response to an audio stimulus, and/or the ability to perceive and/or translate audio content to performance of an action (e.g. a voluntary oculomotor response within the device, a verbal or physical response, or the like). In accordance with some embodiments, earpieces comprising speakers may be retractable, thereby facilitating transport and deployments, and/or improving the portable, compact, and/or ergonomic nature of the HMD.

In accordance with yet other embodiments, an HMD may provide various additional or alternative sensors and/or components for facilitating various other forms of assessments. For instance, earpiece structures may further facilitate, for instance, caloric assessments through the provision of hot or cold fluids and/or various heating elements or other sensors. In accordance with another example, the HMD comprises an ultrasound or like device (e.g. a transcranial Doppler ultrasound device) to evaluate blood flow around the brain of a user. In accordance with some embodiments, such components may be disposed in, on, or in a coupled configuration with the external or outer assembly of the HMD, although it will be appreciated that such aspects may be included within the HMD, for instance within an external casing of the HMD.

In accordance with some embodiments, the HMD comprises one or more position or motion sensors operable to measure, for instance, user movement during assessment. For example, various cognitive impairment assessments known in the art incorporate data related to user head motion. Such data, including, for example, head motion of the subject when asked to track a moving target with their eyes while not moving their head, or a motion pattern observed as a target on which the subject should focus is presented while the user is to rotate their head to one side or the other (or upwards/downwards), may be of avail to a number of assessments. In addition or as an alternative to being sensed as a direct measure of a risk of an impairment (e.g. TBI), and in accordance with some embodiments, such head motion may further be used to provide control data, for instance to normalise data related to oculomotor function, or to correct or eliminate incorrect data for a variety of tests. Accordingly, such data may have standalone value in a cognitive assessment, and/or may serve any one or more of a variety of purposes, in accordance with various embodiments. Various embodiments thus relate to the HMD being operable to acquire user motion data via, for instance, an inertial measurement unit (IMU), gyroscope, accelerometer, or like sensor incorporated therein or thereon.

In this example, the HMD 1900 further comprises infrared (IR) light-emitting diodes (LEDs) 130 and embedded cameras 132 as components of a gaze tracking system.

Unlike conventional head-mounted devices as used, for example, in virtual reality applications, the exemplary HMD 1900 provides a user with a direct wide-angled and unrefracted binocular view of a same display screen. In doing so, rather than to have each eye gaze focused on respective typically small screens via respective intervening lenses or lens sets, and having a binocular experience virtually produced through rendering software, both eyes can directly view a same wide screen, thereby invoking a more natural binocular vision scenario and thus facilitating observation and tracking of a more accurate oculomotor response (i.e. via gaze tracking system), resulting in a more accurate cognitive impairment screening. Indeed, in the exemplary HMD 1900 of this particular embodiment, the user's immersive viewing experience accommodates a wide horizontal field of view. The provision of an unrefracted wide-angle field of view provides, amongst other advantages, for a greater oculomotor range of motion when conducting different vision-based cognitive impairment tests, such as a wide-angle range for smooth pursuit or Optokinetic Nystagmus (OKN), e.g. including tests such as where a visual stimulus is smoothly tracked across the display, or again where a translating pattern of light and dark lines is rendered across the display and a tracking response thereto is monitored. Indeed, for this latter example, a wide-angle field of view is necessary to successfully observe, for instance, a corresponding cognitive impairment, and otherwise generally unachievable using a focused line of sight display.

In this particular embodiment, the display 106 is mounted at the far end of a viewing tunnel, or like structure provided in part by the inner enclosure 120, which immerses the user's gaze to the display 106 and blocks out any external stimuli, while allowing for operation of the eye/gaze tracking system. The mechanically actuated visual stimulus described above is selectively deployed to operate within this viewing tunnel. Indeed, the minimalist viewing tunnel, devoid of any intervening refractive optics, is adapted to minimise any luminous reflections or artefacts and thus, casts the user in a mostly darkened environment where they can focus exclusively on the test stimulus. In some embodiments, an interior surface of the viewing tunnel is provided as an amorphous surface, thereby further reducing internal reflections and visual distractions. For example, the amorphous surface may include, but is not limited to, an amorphous and generally black textured or rugged plastic or like surface, a lined or stretched fabric, opaque stocking, or the like. Accordingly, upon resting the face-resting surface against the user's face around their eyes, a direct, unobstructed and unrefracted wide-angle field of view to the display 106 is provided and confined to the immersive viewing tunnel.

As described above, the device 1900 further comprises, in addition to the display screen 106, a vergence testing feature such as that provided by the mechanically actuated visual stimulus. In this example, the mechanically-actuated visual stimulus guides the user's gaze toward and away from the display 106 in testing, for example, a near point of convergence. For example, a user manifesting a roughly 4 mm near point of convergence will screen out as likely healthy, whereas one manifesting a near point of convergence greater than 8 mm will screen out as likely exhibiting some signs of cognitive impairment. Any anomalous oculomotor responses may also be detected and observed by the HMD 1900.

As further described below, various approaches may be taken to implement a cognitive impairment test via device 1900. One or more stored and user or administrator-guiding tests may be executed locally from memory, whereby a screening result (for each test or overall) may be output locally via the onboard indicator and/or communicated locally or remotely to a corresponding device and user interface, either way guiding the user or caretaker in deciding whether further care or testing may be required. Similarly, an external interface, either provided via a locally or remotely implemented user interface, may provide greater testing control, such as by providing a suite of automated tests (e.g. preset testing visual patterns and/or sequences), and/or manually adjustable or executable tests, for example, where a user dynamically sets visual stimulus thresholds, boundaries, sizes, speeds, ranges, or the like, or again, manually controls displacement, translation and/or positioning of various visual stimuli. In the later example, for instance, an operator could invoke a touchscreen interface replicating the display such that, via touch control, an operator may directly control a location and displacement of a rendered visual stimulus, thus more closely replicating traditional hand/finger tests performed in the field or in clinic.

As briefly described above, user motion (e.g. head or device motion) may be of value in the assessment of a potential user condition (e.g. a concussion). Moreover, greater value may be extracted from user head motion when the head is less hindered by, for instance, the weight of the HMD 1900. For instance, a particularly heavy headset may dampen or otherwise impact head motion, thereby affecting assessment of a potential cognitive impairment. Further, it may not be desirable to encumber or put undue stress on the head or neck of an individual suspected of an injury like a concussion. Accordingly, and in accordance with some embodiments, an HMD may comprise a means for supporting a portion of its weight, such that unnecessary weight is not placed on a patient's head or neck. For example, and in accordance with one embodiment, an assessment device may comprise one or more handles that may be grasped by a hand(s) of the patient or practitioner conducting an assessment, or one or more legs (e.g. a tripod) to support at least a portion of the weight of the system.

Different embodiments as considered herein may include, for example, a standalone HMD unit, a two-part HMD, or other potential head-mounted or face-resting configurations, which may include a complementary digital display (e.g. widescreen, light-field or other like display technologies), for example as previously described in Applicant's co-pending International Application No. PCT/US2022/013564. As detailed in that co-pending application, various embodiments may relate to an assessment device or method in which an HMD may be in communication either wirelessly or by a wired connection to external computational systems. For example, an HMD as considered herein may comprise any necessary on-board computation resources or hardware components to enable wireless communication using known protocols (e.g. Bluetooth™, internet-based, or like communication protocols) with external devices, such as a medical practitioner laptop or desktop computer system or smartphone. Similarly, the HMD may be coupled to such a resource via a wired or wireless connection, and may comprise any necessary components known in the art to enable same. In multi-component systems, such circuitry and/or electronic components may be disposed on, for instance, a load-bearing portion so to minimise undue weight on the user. In either case, it will be appreciated that such connectivity may enable, for instance, communication of assessment data, which may include, for example, metrics related to eye movement during the assessment, raw or filtered images or video of the user's eyes (e.g. live-streaming of the user's eyes) during assessment, as well as display of assessment stimuli or a representation thereof (e.g. what is displayed via a screen to the user during assessment). In accordance with yet further embodiments, such communication may enable remote control of the assessment from a practitioner. For example, a practitioner may control the presentation of a stimulus manually from a laptop or smartphone, which may be manifested in real time within the device to perform an assessment.

Such visualisation and control functionality may be provided via, for instance, a graphical user interface (GUI) associated with a practitioner device, such as a smart phone, tablet, laptop, or desktop computer. For example, in some cases the GUI displays real-time metrics related to pupil diameter variation, gaze dynamics (e.g. gaze displacement, velocity, acceleration, or the like), as well as any head displacement or orientation. The GUI may further display video of the user's eye(s) from within the HMD, as well as a representation and/or manual control screen of the display screen and/or stimulus as would be seen by the user of the device. Various monitoring parameters may further be displayed and/or editable, such as a camera frame rate (e.g. reported as Hz, number of frames per second, or number of frames rendered per second), number of samples acquired, and/or any target offsets between where a target stimulus is presented relative to where the user is actually looking. Other features, functions or assessment data access may also or alternatively be considered, such as for example, digital access to a listing of assessed patients (i.e. database), and documentation, annotation and/or input recommendations associated therewith.

In accordance with various embodiments, an HMD may be configured to automatically execute assessments (e.g. through the automatic execution of digital instructions to perform the assessment, which may be stored on-board an HMD or accessed from a remote or connected system), or to provide manual control over an assessment by a practitioner. Such assessments may relate to, without limitation, saccade tests (e.g. predictive horizontal saccade tests, non-predictive horizontal saccade tests, predictive vertical saccade tests, and/or non-predictive vertical saccade tests), smooth pursuit tests (e.g. predictive or non-predictive horizontal or vertical smooth pursuit tests), reaction time tests, subjective visual tests, optokinetic nystagmus (OKN) tests (e.g. OKN horizontal and/or vertical tests), or vergence tests, to name a few.

As noted above, various assessments can rely on the rendering of stimuli via a display screen within an HMD. As the position of stimuli is varied in up to two dimensions in the plane of the display screen over the course of an assessment, such stimuli may be considered to be dynamic stimuli. However, various embodiments herein described may additionally or alternatively relate to dynamic stimuli that move or appear to move in a third axis (i.e. towards or away from the user), as noted above, without requiring the generation of a light field.

One example of such an assessment is a vergence assessment, as discussed above, provided via a vergence assessment tool disposed within the HMD. A vergence test provides the practitioner with a tool that stimulates the user to move their eyes synchronously and symmetrically in opposite directions. If the motion of the eyes is towards the nose, it is known as convergence, and conversely, if the movement of the eyes is away from the nose and towards the ears, the movement is referred to as divergence. During such an assessment, in addition to the angular orientation of the eyes, the user must be able to adjust the eye's focus at the object located at the different distances, namely, accommodation. These two biological mechanisms work simultaneously to achieve a fast focused image at varying distances. If the movement is abnormal (e.g. asynchronous or with the same angular orientation/motion), the user would be identified as having convergence insufficiency (CI). Common biomarkers of CI include, but are not limited to, blurry vision, diplopia (i.e. double vision), near-sightedness, discomfort, nausea, discomfort, and eye fatigue (which are similarly most commonly observed following a trauma).

In accordance with various embodiments, a vergence test allows the user to look at equidistant axial points in space spanning from a far end of the display (e.g. the end of the display whereat a screen is disposed) to the nose. In some embodiments, this relates to a distance span of approximately 150 mm.

In accordance with some of the embodiments described above, various assessment devices relate to the provision of a dynamic visual stimulus (i.e. one that may by presented as being disposed or moving in 1D, 2D, or 3D within an HMD) via different means to elicit an oculomotor response of a user that is monitored via a gaze tracking system in the HMD for metric monitoring and reporting. Such stimuli may be provided via different means in up to three dimensions, in accordance with different embodiments of an HMD.

For example, in addition to a dynamic stimulus relating to the activation of different pixels on a static 2D display screen, and/or the provision of a 1D array of LEDs sequentially activated within the HMD, various other configurations and components are herein considered that relate to physical displacement of the stimulus itself, such as one presented via a displaceable screen, thereby providing 3D content or stimuli to further assess a patient response thereto presented at various depths. Such embodiments may overcome various drawbacks known to exist with light field, virtual reality (VR), or augmented reality (AR) systems, such as the vergence-accommodation conflict that may lead a user to experience fatigue, discomfort, or nausea.

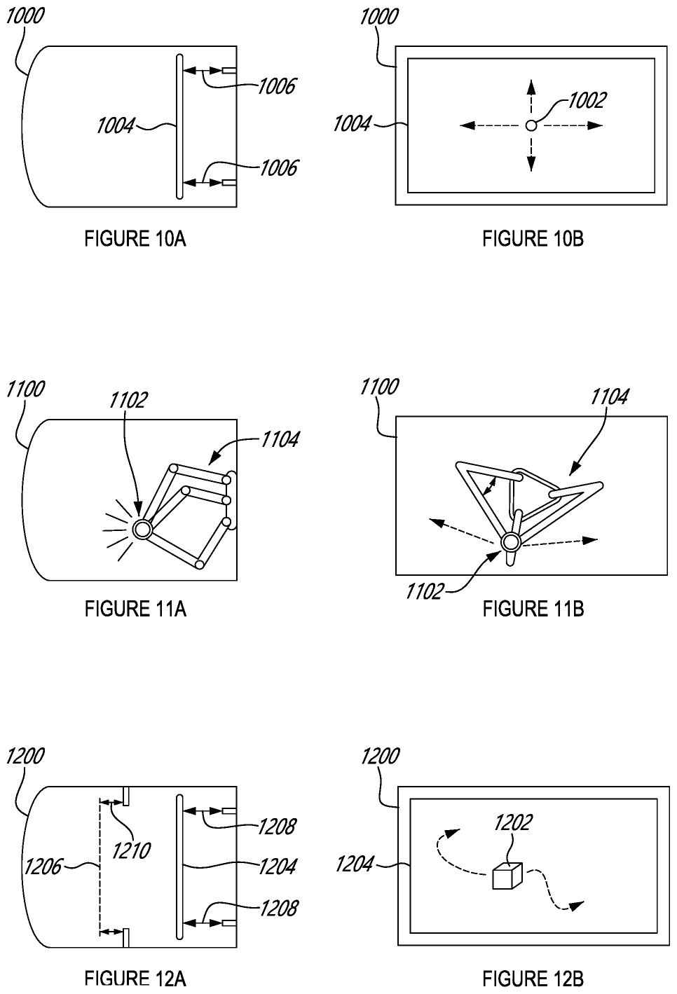

At least in part to this end, FIGS. 10A to 12B show various exemplary configurations for providing a dynamic visual stimulus to perform an assessment, in accordance with different embodiments. For these exemplary embodiments, FIGS. 10A, 11A, and 12A are schematics representing right side sectional views of different assessment systems comprising respective exemplary dynamic stimuli, while FIGS. 10B, 11B, and 12B are schematics representing front views (i.e. from the point of view of a user) corresponding to the side sectional views of FIGS. 10A, 11A, and 12A, respectively. It will be appreciated that these schematics are not necessarily presented to scale, and that that only some components of exemplary assessment systems are shown for clarity and illustrative purposes.

As schematically illustrated in FIGS. 10A and 10B, one exemplary embodiment of an assessment system 1000 comprises a dynamic visual stimulus 1002 that is renderable via a display screen 1004 (e.g. a pixelated display screen 1004). In this non-limiting example, the display screen 1004 is coupled with the assessment system 1000 via actuators 1006 that are operable to translate or displace the screen 1004 closer to a user of the system (i.e. to the left in FIG. 10A, or out of the page in FIG. 10B), and/or further away from a user of the system (i.e. to the right in FIG. 10A, or into the page in FIG. 10B). Accordingly, such a system may be operable to perform, for instance, a vergence test. For example, a stimulus 1002 on a which a user focuses may be rendered on the screen 1004, and the screen 1004 may be translated towards the user while their eyes are monitored using an eye tracking system.

It will be appreciated that, in accordance with some embodiments, a dynamic visual stimulus may be re-rendered or otherwise adjusted during translation of the display screen 1004. For example, one embodiment relates to rendering a variably-sized stimulus (e.g. a visual stimulus that increases or decrease in size, a stimulus that changes shape or brightness, or the like) before, during, or after an assessment. Such a variably-sized stimulus may, for instance, improve a perception of an advancing or retreating stimulus during a vergence test, and/or improve a user comfort level while viewing to assist in mediating and sensory conflicts that a user may experience during assessments.

Furthermore, such a system may be operable to perform other vision-based assessments in addition to vergence tests. For example, the display screen 1004 may be operable to render a dynamic stimulus 1002 that appears to be moving in a plane characterised by the display screen 1004. For example, a saccadic assessment may be performed by sequentially rendering the dynamic stimulus at different regions of the display screen while the user's eyes are monitored. Similarly, a pursuit test may be performed by monitoring the user while the stimulus 1002 is rendered such that it appears to be moving across the screen 1004. It will be appreciated that such tests may be performed while the display screen is translated or displaced via the actuators 1006, thus providing a target stimulus 1002 that is moved, or has the appearance of moving, in three dimensions, in accordance with various embodiments.

In accordance with various embodiments, a dynamic visual stimulus provided by a display screen (e.g. screen 1004) as a rendered light source in an otherwise dark environment. For instance, and in accordance with one embodiment, all pixels of the display screen 1004 may be inactive (i.e. dark), while the stimulus is moved in up to three dimensions via one or more of a rendering sequence and activation of one or more actuators 1006 coupled to the display screen 1004.

In accordance with another embodiment, FIGS. 11A and 11B schematically show an assessment system 1100 comprising a stimulus 1102 that is dynamically adjustable in one, two, and/or three dimensions via a translation device 1104. In accordance with different embodiments, the translation device 1104 may comprise one or more actuators 1106, a robotic arm, a delta robot 1110, or the like, such that the dynamic stimulus may be translated, for instance, towards and/or away from a user. For example, the translation device may comprise an actuator operable to translate a stimulus along a track towards a user while the user's eyes are monitored, thereby performing a vergence test. Additionally, or alternatively, the translation device 1104 may comprise a plurality of actuators 1106 such that the stimulus 1102 may be translated in a plane (e.g. in two dimensions at a designated distance from the user's eyes) to perform a conventional pursuit assessment.

The translation device 1104 (e.g. a delta robot 1104) may similarly be operable to rapidly reposition the stimulus 1102 such that the stimulus 1102 is perceived to have appeared at a new location, thereby enabling a saccade assessment. In accordance with some embodiments, the translation device 1104 may be operable to translate the stimulus 1102 smoothly and/or rapidly in three dimensions, thereby enabling various other assessments, or combinations thereof.

In accordance with different embodiments, the dynamic stimulus 1102 may comprise different elements or components. For example, a dynamic stimulus 1102 of a first embodiment may comprise an LED or other light source that is translatable in three dimensions via a delta robot 1104. In accordance with another embodiment, the dynamic stimulus may comprise a display screen that is translatable via a translation device 1104. For example, a small display screen (e.g. 1 cm×1 cm, 1″×1″, or the like) may be coupled with a robotic arm such that a stimulus 1102 may be rendered by the display screen to be tracked by the user while it is translated in up to three dimensions. Accordingly, through the employ of a small screen, a rendered stimulus 1102 may be physically translated in up to three dimensions, without requiring re-rendering or updating of pixel values to simulate movement. By comparison, the stimulus 1002 of FIGS. 10A and 10B may be rendered and/or moved on a display screen 1004 in two dimensions (i.e. the x-y plane) by way of refreshing displayed content, while the screen 1004 itself is translated in a third dimension toward or away from the user (i.e. the z-dimension or axis).

It will be appreciated that, in accordance with various embodiments, the assessment device 1100 may provide an isolated (i.e. dark) environment in which to perform a cognitive assessment. For example, the load-bearing portion of an assessment device may substantially eliminate ambient light during performance of an assessment. Accordingly, a dynamic stimulus may comprise a light source (e.g. an LED, activated pixels of a display screen, or the like) that is readily tracked by the user. Conversely, various other embodiments relate to the provision of a stimulus in a lit environment, such that a stimulus 1102 that is not inherently a light source may be seen and tracked by a user during an assessment. For instance, any object that is not a light source may be translated by a delta robot in ambient conditions in up to three dimensions while a user's gaze is monitored to perform a cognitive assessment. As described above, various embodiments of an assessment system comprise a stimulus that is dynamically adjustable in one, two, and/or three dimensions via a translation device. In the exemplary embodiment of, for instance, FIGS. 11A and 11B, the translation device 1104 comprises one or more actuators 1106, a robotic arm, a delta robot 1110, or the like, such that the dynamic stimulus 1102 may be translated, for instance, towards and/or away from a user. However, it will be appreciated that the dynamic stimulus 1102 and translation device 1104 of FIGS. 11A and 11B relate to non-limiting examples of components and/or mechanisms for providing a dynamic stimulus in the context of a head-mountable device.

Furthermore, it will be appreciated that, in accordance with various embodiments, a cognitive assessment system may reduce user discomfort by providing an assessment in conditions that are more natural to the user than are provided by, for instance, AR or VR systems. For example, a known challenge with VR systems is the occurrence of nausea or other symptoms as the user experiences conflicting sensory stimuli. It is herein contemplated that such conflicting stimuli may arise from as seemingly benign sources as ambient light, which may inevitably enter even an ‘isolated’ assessment system 1100, reflecting or otherwise interacting with system components, such as a display screen or pixels thereof, which are then perceived with negative effects by the user. Accordingly, various embodiments address such challenges through the use of alternative stimuli to those conventionally used in, for instance, saccade or pursuit assessments. For example, while conventional systems may employ relatively large static display screens to render content in two dimensions, some embodiments herein disclosed relate to the provision of a stimulus using a small display screen that has a majority of pixels activated to provide a visual stimulus. Accordingly, such embodiments may comprise a reduced amount of non-active pixels, glass, or the like, from which light may inadvertently be directed at the user and potentially cause discomfort. Similarly, a dynamic stimulus 1102 comprising a single, or a small number, of LEDs, or other light sources (e.g. fibre optics for guiding light) may be more benignly perceived by a user, even in the presence of stray light entering the system.

With reference now to FIGS. 12A and 12B, a further embodiment of an assessment system 1200 may comprise a light field display as a means of providing a dynamic stimulus 1202 for an assessment. In this example, the light field display comprises a pixelated display screen 1204 and a light field shaping layer (LFSL) 1206. In accordance with different embodiments, a LFSL may comprise, without limitation, a microlens array (MLA), a pinhole array, a parallax barrier, or other means known in the art, or a combination thereof, for shaping or governing a light field. Accordingly, it will be appreciated that various processes (e.g. ray tracing) and processing resources enabling the generation of a light field for performing a cognitive assessment may similarly be employed in accordance with various embodiments herein contemplated.

In the embodiment of FIGS. 12A and 12B, a dynamic stimulus 1202 for performing a cognitive assessment may be rendered via a combination of the display screen 1204 and LFSL 1206. While it will be appreciated that such systems may be operable to provide a 3D stimulus, and/or one that may be rendered to be perceived by a user as originating from one or more of a plurality of depth planes in, for instance, a vergence test, various embodiments may further relate to the provision of a dynamic stimulus via a translating or translated display screen 1204, and/or a translating or translated LFSL 1206. For example, one embodiment of an assessment system 1200 comprises a display screen 1204 that is coupled with the assessment system 1200 via one or more actuators 1208 operable to displace the screen relative to a user of the system and/or the LFSL 1206. Similarly, the LFSL 1206 may be displaceable via one or more actuators 1210 or like systems to translate the LFSL 1206 relative to the display screen 1204 and/or user. Accordingly, and in accordance with various embodiments, a cognitive assessment system 1200 operable to generate a light field may be operable to not only perform a vergence assessment, but may be operable to do so over, for instance, a wider range of rendered optotypes at a designated assessment resolution.

For example, a static light field shaping system may be operable to render a stimulus to be perceived at depth planes corresponding to a particular range of dioptric corrections that is limited by system components (e.g. MLA pitch and/or focal length, screen resolution, a spacing between the LFSL and screen, or the like). Conversely, a dynamically translatable display screen 1204 and/or LFSL 1206 may enable a wider and/or different range of dioptric corrections achievable for a system otherwise comprising the same components. Accordingly, and in accordance with various embodiments, a dynamic stimulus rendered via a dynamically adjusted display screen 1204 and/or LFSL 1206 may provide for a greater range of depth planes in a vergence assessment than would be accessible with a conventional static light field display for, for instance, a designated resolution of displayed content. In accordance with yet other embodiments, adjustment or dynamic displacement of a display screen 1204 and/or LFSL 1206 of a light field display may be employed to mitigate, for instance, vergence-accommodation conflicts that may hinder or otherwise give rise to user discomfort during a cognitive impairment assessment.

It will be appreciated that a dynamic stimulus in a light field-based assessment device 1200 may be rendered in different positions of the light field display to perform, for instance, saccade or pursuit tests, as described above. Further, various light field-based systems and methods herein disclosed may comprise integrated vision correction, and may enable correction of rendered content in accordance with a corrective eye prescription for a test or set of designated tests. Such corrections may be applied in addition to or alternatively to dioptric changes inherent in some tests (e.g. amplitude of accommodation tests). For example, and in accordance with various embodiments, a light field-based cognitive assessment may comprise the presentation of content to the subject in accordance with a perception adjustment designated so to accommodate a reduced visual acuity of the subject. That is, a conventional cognitive assessment targeting the oculomotor system may comprise presenting content (e.g. a test for assessing saccadic movement, smooth pursuit, etc.) at a fixed distance from the subject's eye(s) (e.g. from a 2D tablet screen or computer monitor), requiring a subject having a reduced visual acuity (e.g. farsighted, nearsighted, or the like) to wear prescriptive lenses to properly view the content. Conversely, various embodiments herein described relate to the operation of a light field assessment system 1200 for the presentation of content having a dioptric correction or optotype applied thereto (e.g. +3.0 D, −4.25 D, etc.). Accordingly, various embodiments allow the subject to properly view content without glasses or another form of corrective lenses, which would otherwise hinder the assessment by, for instance, interfering with eye tracking, inhibiting proper alignment of the device on the subject's face, or the like. Such content adjustments may be presented in addition to, for instance, dioptric corrections or image depth plane adjustments inherent in, for instance, a near point of accommodation or vergence assessment.

It will further be appreciated that while the application of such dioptric corrections may improve a quality or outcome of cognitive assessment tests, the dioptric correction required for a subject to clearly see displayed content may itself constitute a diagnostic test, in accordance with one embodiment. For example, a cognitive impairment assessment device 1200 may be operable to assess the visual acuity of a user through, for instance, the display of different optotypes. If a subject is observed to not exhibit a prior baseline of visual acuity, they may be exhibiting signs of a cognitive impairment. In accordance with various embodiments, an assessment system (e.g. assessment system 1000, 1100, 1200) may comprise dark materials so to provide an isolated environment in which to perform an assessment. For example the inner casing of an assessment device may comprise a dark inner lining of a non-reflective material, thereby minimising stray light that may distract a user during an assessment, or provide a user with discomfort, as described above. It will be appreciated that any other system components, such as processing resources (e.g. one or more digital data processors operable to execute digital instructions for performing ocular cognitive impairment assessment via a display screen, actuators, eye tracking systems, or the like), sensors, electronics, power sources, or the like, may be similarly encased and/or concealed within an assessment system.

It will further be appreciated that various embodiments relate to assessment devices comprising wireless functionality. For example, some embodiments relate to the provision of assessments as described above, while further providing assessment guidance via a display screen (e.g. screen 1004, or 1204) before, during, or after assessment. For example, an assessment system may comprise telepresence functionality to display a remote medical practitioner on a display screen during assessment as a picture-in-picture window. Accordingly, these and other embodiments herein contemplated may further comprise a microphone or like component to, for instance, communicate and/or record user responses or feedback during an assessment.

The following description relates to various hardware specifications and configurations of an HMD for performing oculomotor assessments, in accordance with one embodiment, beyond the mechanical arrangements described above for the mechanical actuation of a visual stimulus.

As described above, an HMD may communicate with a practitioner device through either a wired or wireless connection. In the case of a wired connection to, for instance, a practitioner laptop, the HMD may comprise the following non-exhaustive list of components, in accordance with one embodiment: an infrared-based embedded eye tracker for gaze extraction, an inertial measurement unit, a wide-angle display, a display driver board, a multi-port hub, a development board (e.g. a Teensy™ 3.2 development board), an RGB LED indicator,, and a USB adapter. Such components may interact with a practitioner device operable to execute machine executable instructions (e.g. a digital application) on a practitioner device to perform various assessments.

In embodiments of an HMD operable to perform assessments without a wired connection, the HMD may comprise the components described above, and may further comprise a single-board computer (e.g. an NUC computing device), a touch screen display, a remote control (e.g. a clicker with Bluetooth and/or USB dongle functionality), and/or a wireless keyboard and/or mouse, in accordance with one embodiment.

With respect to hardware components, various aspects may be of consideration, depending on the application at hand. Accordingly, various specifications may be preferred for various embodiments of HMD configurations, and are hereby contemplated. For example, one non-limiting embodiment of an HMD comprises an embedded IR-based eye tracking system for gaze extraction having the following specifications: a tracking frequency of approximately 200 Hz, a field of view greater than approximately 100 degrees, a gaze accuracy of at least approximately 1.0 degrees, a gaze precision of at least approximately 0.08 degrees, a camera latency of approximately 8.5 ms or less, a processing latency of approximately 4 ms or less, an image resolution of approximately 192×192, at least a 5-point calibration method, and is operable in one or more of a stereo-mode, whereby both eye images are extracted to estimate binocular gaze, and a mono-mode, whereby each eye image is extracted to estimate monocular gaze. Such specifications are non-limiting. For example, in accordance with one embodiment, an embedded IR gaze tracking system may comprise cameras operating at 30 Hz with a 400×400 image resolution. In accordance with one embodiment, an embedded IR gaze tracking system comprises a Pupil Labs™ HTC Vive Add-on set.

In accordance with one embodiment, an embedded IMU within an HMD may relate to the following non-limiting specifications, in accordance with one embodiment: a resolution of less than approximately 0.01 degrees, an orientation range corresponding to roll (±180°), pitch (±90°), and yaw (±80°), static and dynamic accuracies of less than approximately 0.5 degrees and 2 degrees, respectively, a data transition rate of up to approximately 400 Hz, a 3-axis accelerometer, a 3-axis magnetometer, and a 3-axis gyroscope. In one embodiment, an embedded IMU is set to operate at a transmission rate of 100 Hz and extracts the Euler angles during the various assessments. In one embodiment, the embedded IMU comprises a Xikaku™ LPMS-B2 model.

In accordance with one embodiment, wide-angle display within an HMD may relate to the following non-limiting specifications, in accordance with one embodiment: a diagonal size of approximately 8.8″, a 1920×480 pixel format, a 218.88×54.72 mm (H×V) display area, an aspect ratio of at least 3:1, a pixel pitch of approximately 0.114×0.114 mm (H×V), a brightness of approximately 600 cd/m², a contrast ratio of approximately 800:1, approximately 16.7M (8 bit) colour numbers, a USB power of 5 V, and a refresh rate of approximately 60 Hz.

In accordance with various embodiments, a multiport hub may comprise various configurations. In one non-limiting embodiment, a multiport hub comprises four USB-A hubs, an HDMI hub, and a USB-C hub.

In order to facilitate the assembly process of an HMD and reduce cabling within the device, as well as to reduce the risk of disconnection, various embodiments relate to the employ of custom-designed PCBs installed within an HMD.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean ‘one and only one’ unless explicitly so stated, but rather ‘one or more.’ All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A head-mountable device for performing an oculomotor assessment of a user, the head-mountable device comprising:
a mechanically displaceable oculomotor stimulus;
an actuation mechanism operable to displace said mechanically displaceable oculomotor stimulus relative to the user so to present said mechanically displaceable oculomotor stimulus to the user in accordance with the oculomotor assessment;
an eye tracking system configured to monitor an oculomotor response of the user to said mechanically displaceable oculomotor stimulus; and
a digital data processor in communication with said eye tracking system and operable to execute digital instructions for recording said oculomotor response to said mechanically displaceable oculomotor stimulus presented in accordance with the oculomotor assessment.

2. The head-mountable device of claim 1, wherein said digital data processor is further in communication with said actuation mechanism and is operable to execute digital instructions for performing the oculomotor assessment by digitally activating said actuation mechanism to present said mechanically displaceable oculomotor stimulus to the user in accordance with the oculomotor assessment.

3. The head-mountable device of claim 1, wherein said actuation mechanism comprises a manually manipulatable actuation mechanism comprising a user-manipulatable component which, upon manual displacement by the user, elicits a corresponding displacement of said mechanically displaceable oculomotor stimulus.

4. The head-mountable device of claim 1, wherein said actuation mechanism is operable to translate said mechanically displaceable oculomotor stimulus in a direction towards or away from the user's eyes.

5. The head-mountable device of any one of claims 1 to 4, wherein the oculomotor assessment comprises one or more of a vergence response assessment, a convergence insufficiency assessment, or an accommodation assessment.

6. The head-mountable device of any one of claims 1 to 5, wherein said mechanically displaceable oculomotor stimulus comprises a fixation stick mechanically coupled with said actuation mechanism.

7. The head-mountable device of any one of claims 1 to 6, wherein said actuation mechanism comprises one or more of an electric motor, a pulley, a belt, a cable, a threaded rod, a hydraulic or pneumatic actuator, or a cog.

8. The head-mountable device of any one of claims 1 to 7, wherein said mechanically displaceable oculomotor stimulus is pivotably coupled with said actuation mechanism to provide said mechanically displaceable oculomotor stimulus in a retracted configuration or an extended configuration.

9. The head-mountable device of claim 8, wherein said actuation mechanism is operable to mechanically configure said mechanically displaceable oculomotor stimulus in one or more of said retracted configuration or said extended configuration.

10. The head-mountable device of claim 8, further comprising a retaining structure configured to maintain said mechanically displaceable oculomotor stimulus in said retracted configuration upon said actuation mechanism displacing said mechanically displaceable oculomotor stimulus to a stimulus retaining position.

11. The head-mountable device of claim 10, wherein said extended configuration is provided by a force upon said actuation mechanism displacing said mechanically displaceable oculomotor stimulus to a stimulus presentation position.

12. The head-mountable device of any one of claims 1 to 11, further comprising a digital display screen viewable by the user, wherein said digital display screen is operable to render a complementary two-dimensional oculomotor stimulus in accordance with a complementary oculomotor assessment.

13. The head-mountable device of claim 12, wherein said mechanically displaceable oculomotor stimulus is retractable to provide the user an unimpeded line of sight to said complementary two-dimensional oculomotor stimulus.

14. The head-mountable device of either one of claim 12 or claim 13, wherein said digital display screen comprises a widescreen display to render said complementary two-dimensional oculomotor stimulus horizontally displaceable in a wide binocular field of view to stimulate a complementary wide field of view oculomotor response thereto in accordance with said complementary assessment.

15. The head-mountable device of any one of claims 1 to 14, further comprising a head-mountable housing that, when mounted against the user's face, defines an internal visual stimulation chambre therein for presenting the oculomotor stimulus.

16. The head-mountable device of any one of claims 1 to 15, further comprising an illumination source to illuminate said mechanically displaceable oculomotor stimulus so to be perceivable by the user.

17. The head-mountable device of either one of claim 15 or claim 16, further comprising an external assessment indicator externally disposed on said head-mountable housing and configured to output an indicator signal representative of one or more of an assessment status or a screening indicator corresponding with a health risk associated with the oculomotor assessment.

18. The head-mountable device of any one of claims 1 to 17, further comprising an operator application digitally executable on a distinct operator device having an operator display screen and a communication interface to the head-mountable device, wherein said operator application comprises digitally executable instructions to render a graphical user interface (GIU) on said digital display screen and receive as input therefrom manual digital control of said mechanically displaceable oculomotor stimulus such that a stimulus displacement of said mechanically displaceable oculomotor stimulus corresponds with a manual displacement entered via said GUI.

19. The head-mountable device of any one of claims 1 to 18, wherein said mechanically displaceable oculomotor stimulus comprises an indicium perceptible to the user and associated with the oculomotor assessment.

20. The head-mountable device of any one of claims 1 to 19, further comprising a displacement sensor configured to monitor a stimulus displacement during the oculomotor assessment.

21. The head-mountable device of claim 20, wherein said displacement sensor comprises a potentiometer.

22. The head-mountable device of any one of claims 1 to 21, wherein said digital data processor is further operable to execute digital instructions for outputting an assessment response signal representative of said oculomotor response.

23. A head-mountable device for performing a vision-based assessment of a user, the head-mountable device comprising:
a head-mountable housing that, when mounted against the user's face, defines an internal visual stimulation chambre therein;
a mechanically displaceable visual stimulus disposed within said internal visual stimulation chambre and configured to be translated relative to said head-mountable housing in accordance with the vision-based assessment; and
an actuation mechanism operable to displace said mechanically displaceable visual stimulus relative to the user so to present said mechanically displaceable visual stimulus to the user in accordance with the vision-based assessment.

24. The head-mountable device of claim 23, further comprising an internal eye tracker to monitor a user eye response to said mechanically displaceable visual stimulus.

25. The head-mountable device of claim 23, wherein said visual stimulus is at least one of manually or electronically actuated.

26. The head-mountable device of claim 23, wherein said actuation mechanism comprises a manually manipulatable actuation mechanism which, upon manual displacement, elicits a corresponding displacement of said mechanically displaceable visual stimulus.

27. The head-mountable device of claim 23, wherein said actuation mechanism is operable to translate said mechanically displaceable visual stimulus in a direction towards or away from the user's eyes.

28. The head-mountable device of claim 27, wherein the vision-based assessment comprises one or more of a vergence response assessment, a convergence insufficiency assessment, or an accommodation assessment.

29. The head-mountable device of any one of claims 23 to 28, wherein said mechanically displaceable visual stimulus comprises a fixation member mechanically coupled with said actuation mechanism.

30. The head-mountable device of claim 29, wherein said fixation member is retractably coupled to said actuation mechanism to provide said mechanically displaceable visual stimulus in a retracted configuration or a deployed configuration as the actuation mechanism is actuated.

31. The head-mountable device of claim 30, further comprising a retaining structure configured to maintain said mechanically displaceable visual stimulus in said retracted configuration upon said actuation mechanism displacing said mechanically displaceable visual stimulus to a stimulus retaining position.

32. The head-mountable device of claim 31, wherein said deployed configuration is provided by a force upon said actuation mechanism displacing said mechanically displaceable visual stimulus to a deployed stimulus presentation position.

33. The head-mountable device of any one of claims 23 to 28, further comprising a digital display screen mounted within said housing for viewing by the user, wherein said digital display screen is operable to render a complementary two-dimensional vision-based stimulus.

34. The head-mountable device of claim 33, wherein said mechanically displaceable vision-based stimulus is retractable to provide the user an unimpeded line of sight to said complementary two-dimensional vision-based stimulus.

35. The head-mountable device of either one of claim 33 or claim 34, wherein said digital display screen comprises a widescreen display to render said complementary two-dimensional vision-based stimulus horizontally displaceable in a wide binocular field of view to stimulate a complementary wide field of view response thereto in accordance with said complementary assessment.

36. The head-mountable device of any one of claims 23 to 35, further comprising a displacement sensor configured to monitor a stimulus displacement during the vision-based assessment.

37. The head-mountable device of claim 36, wherein said displacement sensor comprises a potentiometer.

38. A video-nystagmograph (VNG) wearable by a user, comprising:
a head-mountable digital display for selectively rendering visual content to the user in accordance with a designated ocular test;
a displaceable visual stimulus selectively displaceable toward and away from said digital display in providing a variable depth visual stimulus to the user in accordance with a complementary ocular test; and
an eye tracker.

39. The VNG of claim 38, further comprising a head-mountable housing that, when mounted against the user's face, defines an internal visual stimulation chambre therein, at the end of which is disposed said digital display and within which said displaceable visual stimulus is displaceable.

40. The VNG of claim 38, further comprising an actuation mechanism operable to mechanically displace said displaceable visual stimulus relative to the user.

41. The VNG of claim 40, wherein said actuation mechanism comprises at least one of a manually or an electronically actuated mechanism.

42. The VNG of claim 38, wherein said complementary ocular test comprises one or more of a vergence response assessment, a convergence insufficiency assessment, or an accommodation assessment.

43. The VNG of any one of claims 38 to 42, wherein said displaceable visual stimulus comprises a fixation member mechanically displaceable relative to the user.

44. The VNG of claim 43, wherein said fixation member is selectively retractable and deployable.

45. The VNG of any one of claims 38 to 42, further comprising a displacement sensor configured to monitor a stimulus displacement during said complementary ocular test.

46. A method of performing an oculomotor assessment of a user, the method comprising:
providing the user with the head-mountable device of any one of claims 1 to 45;
via said actuation mechanism. displacing said mechanically displaceable oculomotor stimulus in accordance with the oculomotor assessment.
